(19) United States
(12) Patent Application Publication
(10) Pub. No.: US 2022/0340843 A1
(43) Pub. Date: Oct. 27, 2022

(54) POLYPEPTIDES COMPRISING AT LEAST TWO CARBOHYDRATE BINDING DOMAINS

(71) Applicant: Novozymes A/S, Bagsvaerd (DK)

(72) Inventors: Lone Baunsgaard, Helsingoer (DK); Michael Lynge Nielsen, Birkeroed (DK); Kenneth Jensen, Oelsted (DK); Hiroshi Teramoto, Chiba (JP); Keiichi Ayabe, Konakadaicho (JP)

(73) Assignee: Novozymes A/S, Bagsvaerd (DK)

(21) Appl. No.: 17/763,592

(22) PCT Filed: Oct. 1, 2020
(86) PCT No.: PCT/EP2020/077452

§ 371 Date: Mar. 24, 2022

(30) Foreign Application Priority Data
Oct. 3, 2019 (EP) 19201271.4

(51) Int. Cl. C11D 3/386; C12N 9/42; C11D 11/00

(52) U.S. Cl. CPC C11D 3/38636; C12N 9/2437; C11D 11/0017; C07K 2319/02; C07K 2319/50

(57) ABSTRACT

Disclosed are fusion polypeptides comprising at least two carbohydrate binding modules (CBMs), wherein the polypeptide has carbohydrate binding activity. Also disclosed is the use of such polypeptides for reducing the wrinkles in laundry, as well as detergent compositions and laundry booster compositions comprising the same. Also disclosed are polynucleotides encoding the variants, nucleic acid constructs, vectors, and host cells comprising the variants and methods of making the variants.

This application contains a Sequence Listing in computer readable form. The computer readable form is incorporated herein by reference.

### FIELD OF THE INVENTION

The present invention relates to polypeptides having carbohydrate binding activity, polynucleotides encoding the variants, methods of producing the variants, and methods of using the variants.

### BACKGROUND OF THE INVENTION

Laundering of textiles is common activities in normal household activities. When clothes have been used it is typically laundered in order to remove dirt and refresh the clothes before it is used again. Most used laundry processes involved washing in an aqueous detergent solution followed by one or more rinses and subsequent drying.

However, it is also commonly experienced that clothes and textiles becomes wrinkled during laundry, and the washed clothes get a wrinkled, less appealing appearance.

It is desirable to reduce the amount of wrinkles formed during laundry of clothes or textiles.

### SUMMARY OF THE INVENTION

The invention provides fusion polypeptides comprising at least two carbohydrate binding modules (CBMs), wherein the polypeptide has carbohydrate binding activity.

### Definitions

As used herein, the singular forms “a”, “an”, and “the” are intended to include the plural forms as well, unless the context clearly indicates otherwise.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Anti-wrinkle and/or anti-crease and/or reducing wrinkle and/or wrinkle reduction: In the context of the present invention, the terms “crease” and “wrinkle” and related terms, such as “anticrease,”“anti-wrinkle,” “reducing wrinkle,” and “wrinkle reduction” refer to non-permanent deformations in fabrics, such as fabrics and textiles, which can be removed by flattening at elevated temperature and moisture (e.g. by ironing). The terms are used interchangeably herein.

Bacterial: In the context of the present invention, the term “bacterial” in relation to polypeptide or carbohydrate binding module refers to a polypeptide encoded by and thus directly derivable from the genome of a bacteria, where such bacteria has not been genetically modified to encode said polypeptide, e.g. by introducing the encoding sequence in the genome by recombinant DNA technology. In the context of the present invention, the term “bacterial carbohydrate binding module” or “carbohydrate binding module obtained from a bacterial source” or “polypeptide is of bacterial origin” thus refers to a polypeptide encoded by and thus directly derivable from the genome of a bacterial species, where the bacterial species has not been subjected to a genetic modification introducing recombinant DNA encoding said polypeptide. Thus, the nucleotide sequence encoding the bacterial polypeptide is a sequence naturally in the genetic background of a bacterial species. A sequence encoding a bacterial polypeptide may also be referred to a wildtype (or parent). The bacterial polypeptide e.g. bacterial carbohydrate binding module also includes naturally occurring polypeptides modified by, e.g., truncation to obtain the portion of the molecule of interest. A bacterial polypeptide includes recombinant produced wild types, as well as synthetically produced peptides. In a further aspect, the invention provides polypeptides substantially homologous to a bacterial polypeptide. In the context of the present invention, the term “substantially homologous” denotes a polypeptide having carbohydrate binding activity which is at least 80%, preferably at least 85%, more preferably at least 90%, more preferably at least 95%, even more preferably at least 96%, 97%, 98%, and most preferably at least 99% identical to the amino acid sequence of a selected bacterial polypeptide.

Carbohydrate binding module: The term “carbohydrate binding module” as used herein refers to the is independent portion of a polypeptide having a contiguous amino acid sequence with a discreet fold and carbohydrate-binding activity. See, e.g., cazy.org/Carbohydrate-BindingModules. While CBMs are often naturally occurring within larger enzymes (typically connected via a linker region to one or more catalytic domains), the term as used herein refers to the independent module. A CBM in its naturally occurring form may be located at the N-terminus, C-terminus, or at an internal position of a polypeptide, and as used herein may be a truncation of its naturally occurring form. Some CBMs are known to have specificity for cellulose.

Exemplary CBM families useful according to the invention are those of CBM family 1, 4, 17, 28, 30, 44, 72 and 79. Again, with reference to cazy.org/Carbohydrate-Binding-Modules, CBM Family 1 includes modules of approximately 40 residues found almost exclusively in fungi. The cellulose-binding function has been demonstrated in many cases, and appears to be mediated by three aromatic residues separated by about 10.4 angstrom and which form a flat surface. CBM family 4 includes modules of approximately 150 residues found in bacterial enzymes. Binding of these modules has been demonstrated with xylan, beta-1,3-glucan, beta-1,3-1,4-glucan, beta-1,6-glucan and amorphous cellulose but not with crystalline cellulose. CBM family 17 includes modules of approximately 200 residues. Binding to amorphous cellulose, cellooligosaccharides and derivatized cellulose has been demonstrated. Regarding CBM family 28, the module from the endo-1,4-glucanase of Bacillus sp. 1139 binds to non-crystalline cellulose, cellooligosaccharides, and β-(1,3)(1,4)-glucans. For CBM Family 30, binding to cellulose has been demonstrated for the N-terminal module of Fibrobacter succinogenes CeIF. The C-terminal CBM44 module of the Clostridium thermocellum enzyme has been demonstrated to bind equally well cellulose and xyloglucan. CBM Family 72 includes modules of 130-180 residues found at the C-terminus glycoside hydrolases from various families, sometimes as tandem repeats. The CBM72 found on an endoglucanase from an uncultivated microorganism was found to bind a broad spectrum of polysaccharides including soluble and insoluble cellulose, beta-1,3/1,4-mixed linked glucans, xylan, and beta-mannan. CBM Family 79 includes modules of approx. 130 residues found so far only in ruminococcal proteins. Binding to various beta-glucans was shown for the R. flavefaciens GH9 enzyme.

In a preferred embodiment, the carbohydrate binding module is not attached to (linked to) a another protein.

As used herein “mixture” or “mixtures” of CBM include blends of polypeptides that are otherwise independently identified, as well as naturally occurring or synthetic constructs of polypeptides. For example, the CBMs useful herein may be present in the former of dimers, trimers, tetramers, and other higher order fusion products, either homologous or heterologous, which may optionally further comprise one or more amino acid linker sequences joining the one or more CBMs.

Catalytic domain: The term “catalytic domain” means the region of an enzyme containing the catalytic machinery of the enzyme. cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a variant. The boundaries of the coding sequence are generally determined by an open reading frame, which begins with a start codon such as ATG, GTG or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a variant of the present invention. Each control sequence may be native (i.e., from the same gene) or foreign (i.e., from a different gene) to the polynucleotide encoding the variant or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a variant.

Detergent components: the term “detergent components” is defined herein to mean the types of chemicals which can be used in detergent compositions. Examples of detergent components are alkalis, surfactants, hydrotropes, builders, co-builders, chelators or chelating agents, bleaching system or bleach components, polymers, fabric hueing agents, fabric conditioners, foam boosters, suds suppressors, dispersants, dye transfer inhibitors, fluorescent whitening agents, perfume, optical brighteners, bactericides, fungicides, soil suspending agents, soil release polymers, anti-redeposition agents, enzyme inhibitors or stabilizers, enzyme activators, antioxidants and solubilizers.

Detergent Composition: the term “detergent composition” refers to compositions that find use in the removal of undesired compounds from items to be cleaned, such as textiles. The detergent composition may be used to e.g. clean textiles for both household cleaning and industrial cleaning. The terms encompass any materials/compounds selected for the particular type of cleaning composition desired and the form of the product (e.g., liquid, gel, powder, granulate, paste, or spray compositions) and includes, but is not limited to, detergent compositions (e.g., liquid and/or solid laundry detergents and fine fabric detergents; fabric fresheners; fabric softeners; and textile and laundry pre-spotters/pretreatment). In addition to containing the enzyme of the invention, the detergent formulation may contain one or more additional enzymes (such as proteases, amylases, lipases, cutinases, cellulases, endoglucanases, xyloglucanases, pectinases, pectin lyases, xanthanases, peroxidases, haloperoxygenases, catalases, nucleases and mannanases, or any mixture thereof), and/or detergent adjunct ingredients such as surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti-corrosion agents, enzyme inhibitors or stabilizers, enzyme activators, transferase(s), hydrolytic enzymes, oxido reductases, bluing agents and fluorescent dyes, antioxidants, and solubilizers.

Expression: The term “expression” includes any step involved in the production of a variant including, but not limited to, transcription, post-transcriptional modification, translation, posttranslational modification, and secretion.

Expression vector: The term “expression vector” means a linear or circular DNA molecule that comprises a polynucleotide encoding a variant and is operably linked to control sequences that provide for its expression.

Fabric improvement: The term “fabric improvement” or “textile improvement” means a benefit not directly related to catalytic stain removal or prevention of re-deposition of soils. Examples of such benefits are anti-backstaining, anti-pilling, anti-shrinkage, anti-wear, anti-wrinkle, improved color appearance, fabric softness, improved shape retention, flame or chemical resistance, anti-odor, anti-UV, water-repellency, anti-microbial, improved association between noncellulosic and cellulosic textiles, improved static control, improved hand or texture, resistance to chemical, biological, radiological or physical hazard, and/or improved tensile strength. Prevention or reduction of dye transfer from one textile to another textile or another part of the same textile is termed anti-backstaining (also termed dye transfer inhibition). Removal of protruding or broken fibers from a textile surface to decrease pilling tendencies or remove already existing pills or fuzz is termed anti-pilling. Coating or reincorporation or smoothing of protruding or broken fibers is also termed anti-pilling. Prevention of or reduction of a decrease in dimensional size is termed anti-shrinkage. Prevention of or repair of abrasion is termed anti-wear. Prevention of wrinkles, recovery of textile from wrinkling, smoothness of seams, and/or retention of creases after repeated home laundering is termed “anti-wrinkle” or anti-crease. Improvement of the textile-softness or reduction of textile stiffness is termed improved fabric softness. Color clarification of a textile, or enhanced colorfastness to laundering, perspiration, light, chlorine and non-chlorine bleach, heat, or light at high temperature is termed improved color appearance. Resistance to dimensional size change or dimensional size change during home laundering is termed improved shape retention. Elevated combustion temperature or resistance to burning or melting at high temperatures is termed flame resistance. Resistance to chemical reactions, solubilization or degradation in the presence of chemical solvents, acid or alkali is termed chemical resistance. Resistance to adsorption or prevention of the retention of odorous compounds, particularly short chain fatty acids or low vapor pressure organic compounds is termed anti-odor. Opacity to and prevention or repair of oxidative damage caused by UV irradiation is termed anti-UV. Decreased retention of water, or resistance to wetting is termed water repellency. Enhanced microbiostatic or microbiocidal properties are termed antimicrobial. An increase in resistance to induced electrostatic charge of a textile, or increase in decay rate of an induced electrostatic charge in a textile is termed improved static control. Resistance to elongation under force or augmentation of breaking force is termed improved tensile strength.

First-wash: The term “first-wash” means showing improvement or performance benefit effect already during or in the first wash or first wash and dry or first dry, and is not dependent on one or more subsequent wash step or wash and dry steps in order to achieve the benefit.

Fragment: The term “fragment” means a polypeptide having one or more (e.g., several) amino acids absent from the amino and/or carboxyl terminus of the carbohydrate binding module, or fusion polypeptide comprising the same; wherein the fragment has carbohydrate binding activity.

Fungal: In the context of the present invention the term “fungal” in relation to polypeptide or carbohydrate binding module refers to a polypeptide encoded by and thus directly derivable from the genome of a fungus, where such fungus has not been genetically modified to encode said polypeptide, e.g. by introducing the encoding sequence in the genome by recombinant DNA technology. In the context of the present invention, the term “fungal carbohydrate binding module” or “carbohydrate binding module obtained from a fungal source” or “polypeptide is of fungal origin” thus refers to a polypeptide encoded by and thus directly derivable from the genome of a fungal species, where the fungal species has not been subjected to a genetic modification introducing recombinant DNA encoding said polypeptide. Thus, the nucleotide sequence encoding the fungal polypeptide may be a sequence naturally in the genetic background of a fungal species. A sequence encoding a fungal polypeptide may also be referred to a wildtype (or parent). The fungal polypeptide e.g. fungal carbohydrate binding module also includes naturally occurring polypeptides modified by, e.g., truncation to obtain the portion of the molecule of interest. A fungal polypeptide includes recombinant produced wild types, as well as synthetically produced peptides. In a further aspect, the invention provides polypeptides substantially homologous to a fungal polypeptide. In the context of the present invention, the term “substantially homologous” denotes a polypeptide having carbohydrate binding activity which is at least 80%, preferably at least 85%, more preferably at least 90%, more preferably at least 95%, even more preferably at least 96%, 97%, 98%, and most preferably at least 99% identical to the amino acid sequence of a selected fungal polypeptide.

Fusion polypeptide: The term “fusion polypeptide” is a polypeptide in which one polypeptide is fused at the N-terminus or the C-terminus of a variant of the present invention. A fusion polypeptide is produced by fusing a polynucleotide encoding another polypeptide to a polynucleotide. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779). A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Hybrid polypeptide: The term “hybrid polypeptide” means a polypeptide comprising domains from two or more polypeptides, e.g., a binding module from one polypeptide and a catalytic domain from another polypeptide. The domains may be fused at the N-terminus or the C-terminus.

Hybridization: The term “hybridization” means the pairing of substantially complementary strands of nucleic acids, using standard Southern blotting procedures. Hybridization may be performed under medium, medium-high, high or very high stringency conditions. Medium stringency conditions means prehybridization and hybridization at 42° C. in 5× SSPE, 0.3% SDS, 200 micrograms/ml sheared and denatured salmon sperm DNA, and 35% formamide for 12 to 24 hours, followed by washing three times each for 15 minutes using 0.2× SSC, 0.2% SDS at 55° C. Medium-high stringency conditions means prehybridization and hybridization at 42° C. in 5× SSPE, 0.3% SDS, 200 micrograms/ml sheared and denatured salmon sperm DNA, and 35% formamide for 12 to 24 hours, followed by washing three times each for 15 minutes using 0.2× SSC, 0.2% SDS at 60° C. High stringency conditions means prehybridization and hybridization at 42° C. in 5×SSPE, 0.3% SDS, 200 micrograms/ml sheared and denatured salmon sperm DNA, and 50% formamide for 12 to 24 hours, followed by washing three times each for 15 minutes using 0.2×SSC, 0.2% SDS at 65° C. Very high stringency conditions means prehybridization and hybridization at 42° C. in 5× SSPE, 0.3% SDS, 200 micrograms/ml sheared and denatured salmon sperm DNA, and 50% formamide for 12 to 24 hours, followed by washing three times each for 15 minutes using 0.2× SSC, 0.2% SDS at 70° C.

Isolated: The term “isolated” means a polypeptide, nucleic acid, cell, or other specified material or component that is separated from at least one other material or component with which it is naturally associated as found in nature, including but not limited to, for example, other proteins, nucleic acids, cells, etc. An isolated polypeptide includes, but is not limited to, a culture broth containing the secreted polypeptide.

Laundering: The term “laundering” relates to both household laundering and industrial laundering and means the process of treating textiles with a solution containing a cleaning or detergent composition of the present invention. The laundering process can for example be carried out using e.g. a household or an industrial washing machine or can be carried out by hand.

Laundry booster: A laundry booster is an additive used to increase the efficacy of a main wash detergent composition.

Mature polypeptide: The term “mature polypeptide” means a polypeptide in its mature form following N-terminal processing (e.g., removal of signal peptide).

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Parent or parent CBM: The term “parent” or “parent CBM” means a carbohydrate binding module to which an alteration is made to produce the enzyme variants of the present invention. The parent may be a naturally occurring (wild-type) polypeptide or a variant or fragment thereof.

Recombinant: The term “recombinant,” when used in reference to a cell, nucleic acid, protein or vector, means that it has been modified from its native state. Thus, for example, recombinant cells express genes that are not found within the native (non-recombinant) form of the cell, or express native genes at different levels or under different conditions than found in nature. Recombinant nucleic acids differ from a native sequence by one or more nucleotides and/or are operably linked to heterologous sequences, e.g., a heterologous promoter in an expression vector. Recombinant proteins may differ from a native sequence by one or more amino acids and/or are fused with heterologous sequences. A vector comprising a nucleic acid encoding a polypeptide is a recombinant vector. The term “recombinant” is synonymous with “genetically modified” and “transgenic”.

Sequence identity: The relatedness between two amino acid sequences or between two nucleotide sequences is described by the parameter “sequence identity”. For purposes of the present invention, the sequence identity between two amino acid sequences may be determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), pref-era-bly version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences may be determined using the Needleman-Wunsch algorithm (Needleman and Wunsch, 1970, supra) as implemented in the Needle program of the EMBOSS package (EM-BOSS: The European Molecular Biology Open Software Suite, Rice et al., 2000, supra), prefer-ably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment).

Textile: The term “textile” means any textile material including yarns, yarn intermediates, fibers, non-woven materials, natural materials, synthetic materials, and any other textile material, fabrics made of these materials and products made from fabrics (e.g., garments and other articles), and is intended to include the term “fabric” as well. The textile or fabric may be in the form of knits, wovens, denims, non-wovens, felts, yarns, and towelling. The textile may be cellulose based such as natural cellulosics, including cotton, flax/linen, jute, ramie, sisal or coir or manmade cellulosics (e.g. originating from wood pulp) including viscose/rayon, cellulose acetate fibers (tri-cell), lyocell or blends thereof. The textile or fabric may also be non-cellulose based such as natural polyamides including wool, camel, cashmere, mohair, rabbit and silk or synthetic polymers such as nylon, aramid, polyester, acrylic, polypropylene and spandex/elastane, or blends thereof as well as blends of cellulose based and non-cellulose based fibers. Examples of blends are blends of cotton and/or rayon/viscose with one or more companion material such as wool, synthetic fiber (e.g. polyamide fiber, acrylic fiber, polyester fiber, polyvinyl chloride fiber, polyurethane fiber, polyurea fiber, aramid fiber), and/or cellulose-containing fiber (e.g. rayon/viscose, ramie, flax/linen, jute, cellulose acetate fiber, lyocell). Fabric may be conventional washable laundry, for example stained household laundry. When the term fabric or garment is used it is intended to include the broader term textiles as well.

Wash cycle: The term “wash cycle” is defined herein as a washing operation wherein textiles are immersed in the wash liquor, mechanical action of some kind is applied to the textile in order to release stains and to facilitate flow of wash liquor in and out of the textile and finally the superfluous wash liquor is removed. After one or more wash cycles, the textile is generally rinsed and dried.

Wild-type: The term “wild-type” in reference to an amino acid sequence or nucleic acid sequence means that the amino acid sequence or nucleic acid sequence is a native or naturally-occurring sequence. As used herein, the term “naturally-occurring” refers to anything (e.g., proteins, amino acids, or nucleic acid sequences) that is found in nature. Conversely, the term “non-naturally occurring” refers to anything that is not found in nature (e.g., recombinant nucleic acids and protein sequences produced in the laboratory or modification of the wild-type sequence).

Wash liquor: The term “wash liquor” is intended to mean the solution or mixture of water and detergents optionally including enzymes used for laundering textiles, for hard surface cleaning or for dishwashing.

### DETAILED DESCRIPTION OF THE INVENTION

Carbohydrate binding modules have demonstrated usefulness for many purposes, including uses for reducing wrinkles and/or providing increased anti-crease properties and/or providing improved ease of ironing and/or providing improved shape retention in a cleaning process of a fabric or textile as described in PCT/EP2019/059510.
The present invention provides non-native multimers of carbohydrate binding modules. The multimers are stable in e.g. detergent compositions including in the presence of protease.

### Polypeptides

In some embodiments, the invention provides a fusion polypeptide comprising at least two carbohydrate binding modules (CBMs) or fragments thereof, wherein the polypeptide has carbohydrate binding activity. In particular, the polypeptide is a non-naturally occurring multimer comprising at least two carbohydrate binding modules or fragments thereof.
The polypeptides may preferably comprise three or more CBMs, such as four or more CBMs, five or more CBMs, six or more CBMs, seven or more CBMs, eight of more CBMs, nine or more CBMs, ten or more CBMs, 11 or more CBMs, 12 or more CBMs, 13 or more CBMs, 14 or more CBMs, 15 or more CBMs, 16 or more CBMs, 17 or more CBMs, 18 or more CBMs, 19 or more CBMs, or even 20 CBMs.
In an embodiment, the at least two CBMs of the polypeptide are the same or different and are each independently selected. For example, the polypeptide can be a heteromultimer, comprising two or more different CBMs. Or, the polypeptide can be a homomultimer, wherein each CBM is the same.
In an embodiment, each CBM is independently selected among CBM family 1, 4, 17, 28, 30, 44, 72 and 79, and mixtures thereof; preferably wherein at least one CBM is a CBM family 1 CBM, and most preferably wherein each CBM is a CBM family 1 CBM.
In an embodiment, at least one CBM, preferably each CBM is derived from a fungus.
In an embodiment, the polypeptide comprises three, four, or five CBMs, each from CBM Family 1; preferably a trimer comprising three different CBMs or a tetramer comprising four different CBMs, each from CBM Family 1.
In an embodiment, the CBMs are joined by a linker region. Where the polypeptide comprises a naturally occurring linker region, the linker region is preferably heterologous to one or more of, and most preferably to each of the CBMs.
Exemplary polypeptides include those wherein each CBM is independently selected among polypeptides having at least 60% sequence identity to one of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 23 e.g. at least 70%, sequence identity, e.g. at least 80% sequence identity, e.g. at least 90% sequence identity; e.g. at least 95%, sequence identity, e.g. at least 96% sequence identity, e.g. at least 97% sequence identity; e.g. at least 98% sequence identity or at least 99% sequence identity, or even 100% sequence identity.
Additional exemplary polypeptides include those wherein each CBM is independently selected from a CBM having the amino acid sequence of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 23 or having an amino acid sequence that deviates from one of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 23 by 1, 2, 3, 4, 5, 6, 7, 8 or 9 substitutions, insertions or deletions.
Preferred polypeptides include those comprising four CBMs having the amino acid sequence of SEQ ID NO: 2, SEQ ID NO: 4, SEQ ID NO: 6 and SEQ ID NO: 23, or having an amino acid sequence that deviates from one of SEQ ID NO: 2, SEQ ID NO: 4, SEQ ID NO: 6 and SEQ ID NO: 23 by 1, 2, 3, 4, 5, 6, 7, 8 or 9 substitutions, insertions or deletions.
In an embodiment, the polypeptide comprises CBMs in the order, from N-terminal to C-terminal: SEQ ID NO: 6-SEQ ID NO: 2-SEQ ID NO: 4, or fragment thereof, optionally joined by a linker region between each CBM. Preferably, the polypeptide is a trimer comprising CBMs in the order, from N-terminal to C-terminal: SEQ ID NO: 6-SEQ ID NO: 2-SEQ ID NO: 4, or fragment thereof, optionally joined by a linker region between each CBM.
In an embodiment, the polypeptide comprises CBMs in the order, from N-terminal to C-terminal: SEQ ID NO: 2-SEQ ID NO: 4-SEQ ID NO: 6, or fragment thereof, optionally joined by a linker region between each CBM. Preferably, the polypeptide is a trimer comprising CBMs in the order, from N-terminal to C-terminal: SEQ ID NO: 2-SEQ ID NO: 4-SEQ ID NO: 6, or fragment thereof, optionally joined by a linker region between each CBM.
In an embodiment, the polypeptide comprises CBMs in the order, from N-terminal to C-terminal: SEQ ID NO: 2-SEQ ID NO: 6-SEQ ID NO: 4, or fragment thereof, optionally joined by a linker region between each CBM. Preferably, the polypeptide is a trimer comprising CBMs in the order, from N-terminal to C-terminal: SEQ ID NO: 2-SEQ ID NO: 6-SEQ ID NO: 4, or fragment thereof, optionally joined by a linker region between each CBM.
In an embodiment, the polypeptide comprises CBMs in the order, from N-terminal to C-terminal: SEQ ID NO: 6-SEQ ID NO: 4-SEQ ID NO: 2, or fragment thereof, optionally joined by a linker region between each CBM. Preferably, the polypeptide is a trimer comprising CBMs in the order, from N-terminal to C-terminal: SEQ ID NO: 6-SEQ ID NO: 4-SEQ ID NO: 2, or fragment thereof, optionally joined by a linker region between each CBM.
In an embodiment, the polypeptide comprises CBMs in the order, from N-terminal to C-terminal: SEQ ID NO: 4-SEQ ID NO: 2-SEQ ID NO: 6, or fragment thereof, optionally joined by a linker region between each CBM. Preferably, the polypeptide is a trimer comprising CBMs in the order, from N-terminal to C-terminal: SEQ ID NO: 4-SEQ ID NO: 2-SEQ ID NO: 6, or fragment thereof, optionally joined by a linker region between each CBM.
In an embodiment, the polypeptide comprises CBMs in the order, from N-terminal to C-terminal: SEQ ID NO: 4-SEQ ID NO: 6-SEQ ID NO: 2, or fragment thereof, optionally joined by a linker region between each CBM. Preferably, the polypeptide is a trimer comprising CBMs in the order, from N-terminal to C-terminal: SEQ ID NO: 4-SEQ ID NO: 6-SEQ ID NO: 2, or fragment thereof, optionally joined by a linker region between each CBM.
In an embodiment, the polypeptide comprises CBMs in the order, from N-terminal to C-terminal: SEQ ID NO: 6-SEQ ID NO: 2-SEQ ID NO: 4-SEQ ID NO: 23, or fragment thereof, optionally joined by a linker region between each CBM. Preferably, the polypeptide is a tetramer comprising CBMs in the order, from N-terminal to C-terminal: SEQ ID NO: 6-SEQ ID NO: 2-SEQ ID NO: 4-SEQ ID NO: 23, or fragment thereof, optionally joined by a linker region between each CBM.
In an embodiment, the polypeptide comprises CBMs in the order, from N-terminal to C-terminal: SEQ ID NO: 2-SEQ ID NO: 4-SEQ ID NO: 6-SEQ ID NO: 23, or fragment thereof, optionally joined by a linker region between each CBM. Preferably, the polypeptide is a tetramer comprising CBMs in the order, from N-terminal to C-terminal: SEQ ID NO: 2-SEQ ID NO: 4-SEQ ID NO: 6-SEQ ID NO: 23, or fragment thereof, optionally joined by a linker region between each CBM.
In an embodiment, the polypeptide comprises CBMs in the order, from N-terminal to C-terminal: SEQ ID NO: 2-SEQ ID NO: 6-SEQ ID NO: 4, or fragment thereof, optionally joined by a linker region between each CBM. Preferably, the polypeptide is a tetramer comprising CBMs in the order, from N-terminal to C-terminal: SEQ ID NO: 2-SEQ ID NO: 6-SEQ ID NO: 4-SEQ ID NO: 23, or fragment thereof, optionally joined by a linker region between each CBM.
In an embodiment, the polypeptide comprises CBMs in the order, from N-terminal to C-terminal: SEQ ID NO: 6-SEQ ID NO: 4-SEQ ID NO: 2-SEQ ID NO: 23, or fragment thereof, optionally joined by a linker region between each CBM. Preferably, the polypeptide is a tetramer comprising CBMs in the order, from N-terminal to C-terminal: SEQ ID NO: 6-SEQ ID NO: 4-SEQ ID NO: 2-SEQ ID NO: 23, or fragment thereof, optionally joined by a linker region between each CBM.
In an embodiment, the polypeptide comprises CBMs in the order, from N-terminal to C-terminal: SEQ ID NO: 4-SEQ ID NO: 2-SEQ ID NO: 6-SEQ ID NO: 23, or fragment thereof, optionally joined by a linker region between each CBM. Preferably, the polypeptide is a tetramer comprising CBMs in the order, from N-terminal to C-terminal: SEQ ID NO: 4-SEQ ID NO: 2-SEQ ID NO: 6-SEQ ID NO: 23, or fragment thereof, optionally joined by a linker region between each CBM.
In an embodiment, the polypeptide comprises CBMs in the order, from N-terminal to C-terminal: SEQ ID NO: 4-SEQ ID NO: 6-SEQ ID NO: 2-SEQ ID NO: 23, or fragment thereof, optionally joined by a linker region between each CBM. Preferably, the polypeptide is a tetramer comprising CBMs in the order, from N-terminal to C-terminal: SEQ ID NO: 4-SEQ ID NO: 6-SEQ ID NO: 2-SEQ ID NO: 23, or fragment thereof, optionally joined by a linker region between each CBM.
In an embodiment, the polypeptide comprises one or more polypeptide as set forth in the Examples.
In an embodiment, the polypeptide has at least 60% sequence identity, e.g., 70% sequence identity, e.g. at least 80% sequence identity, e.g. at least 90% sequence identity; e.g. at least 95%, sequence identity, e.g. at least 96% sequence identity, e.g. at least 97% sequence identity; e.g. at least 98% sequence identity or at least 99% sequence identity, or even 100% sequence identity to the polypeptide of SEQ ID NO: 30, SEQ ID NO: 32, or SEQ ID NO: 38.

The polynucleotide encoding the polypeptide preferably comprises, consists essentially of, or consists of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, or SEQ ID NO: 22.

In some embodiments, the present invention relates to a polypeptide derived from a mature polypeptide of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 23 by substitution, deletion or addition of one or several amino acids in the mature polypeptide of SEQ ID NO: 2. In some embodiments, the present invention relates to variants of the mature polypeptide of SEQ ID NO: 2 comprising a substitution, deletion, and/or insertion at one or more (e.g., several) positions. In one aspect, the number of amino acid substitutions, deletions and/or insertions introduced into the mature polypeptide of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 23 is up to 10, e.g., 1, 2, 3, 4, 5, 6, 7, 8, 9, or 10. In an embodiment, the polypeptide has an N-terminal extension and/or C-terminal extension of 1-10 amino acids, e.g., 1, 2, 3, 4, 5, 6, 7, 8, 9, or 10 amino acids. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.
Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant molecules are tested for carbohydrate binding activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7:127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

In one embodiment of the present invention, the polypeptide having carbohydrate binding activity may according to the present invention be added to a detergent composition in an amount corresponding to 0.001-200 mg of protein, such as 0.005-100 mg of protein, preferably 0.01-50 mg of protein, more preferably 0.05-20 mg of protein, even more preferably 0.1-10 mg of protein per liter of wash liquor.
In one embodiment, the polypeptide having carbohydrate binding activity is joined to another polypeptide used in the laundering process, such as an enzyme. In this embodiment, the amount of polypeptide having carbohydrate binding activity should be calculated based on the weight of the polypeptide having carbohydrate binding activity alone, without the weight of the polypeptide joined thereto.
The CBM, may according to the invention be added during the washing process and in this embodiment, the CBMs are typically incorporated in the detergent composition used for the laundry process. In an alternative embodiment, the CBMs are added during the rinse following the washing process and in this embodiment, the CBMs are typically incorporated in a rinsing aid composition.
In another embodiment, the polypeptide having carbohydrate binding activity is not joined to any other polypeptide.
According to the invention the use of the polypeptide having carbohydrate binding activity can reduce the wrinkles occurring during the laundry process compared with a similar washing process without addition of the polypeptide having carbohydrate activity. The number of wrinkles are according to the invention be assessed using the AATCC (American Association of Textile Chemists and Colorists) test method 124-TM 124 Smoothness Appearance of Fabrics after Home Laundering (https://members.aatcc.org/store/tm124/533/).
According to the invention the score is improved with at least 0.15 units, 0.20 units, 0.25, units, 0.30 units, 0.40 units, preferably at least 0.5 units, preferably at least 0.75 unit, preferably at least 1.0 units, preferably at least 1.25 units, preferably at least 1.5 units, preferably at least 1.75 units, preferably at least 2.0 units or even higher.
According to the invention the fabric improvement can be evaluated by panelist assessment. Panelists are asked to select towel part being the softest and to select T-shirt part being the less creased. After evaluation, distribution is calculated. The softness and anti-crease is indicated with X:Y values, wherein X specifies the % of the panelists preferring real items washed with CBM, and Y specifies the % that prefers real item washed without CBM. The sum of the X and Y values is 100%.
According to the invention, the panelists preferring fabrics washed with CBM vs test panelists preferring fabrics washed without CBM is at least 60:40, preferably at least 70:30, preferably at least 80:20 or preferably at least 90:10. Preferably, the improved softness effect ratio of test panelists preferring fabrics washed with CBM vs test panelists preferring fabrics washed without CBM is at least 60:40, preferably at least 70:30, preferably at least 80:20 or preferably at least 90:10.
The invention is not limited to any particular laundering process but can be applied to any laundering process using laundering equipment as known in the art, such as front loader or top loader washing machines, or even hand wash.
The invention is neither limited by the way the textile is dried after the wash, but the invention can be used in combination with any method for drying the textiles, include line drying or the use of a dryer, such as a tumble dryer.
The invention is not limited to any particular fabric or textile but can be applied to any known textiles such as cotton, PET, rayon, viscose, wool and silk and any blends of these.

### Preparation of Polypeptides

The carbohydrate binding modules described herein can be prepared using any mutagenesis procedure known in the art, such as site-directed mutagenesis, synthetic gene construction, semi-synthetic gene construction, random mutagenesis, shuffling, etc.

Site-directed mutagenesis is a technique in which one or more mutations are introduced at one or more defined sites in a polynucleotide encoding the parent.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually the restriction enzyme that digests the plasmid and the oligonucleotide is the same, permitting sticky ends of the plasmid and the insert to ligate to one another. See, e.g., Scherer and Davis, 1979, Proc. Natl. Acad. Sci. USA 76: 4949-4955; and Barton et al., 1990, Nucleic Acids Res. 18: 7349-4966.

Site-directed mutagenesis can also be accomplished in vivo by methods known in the art. See, e.g., U.S. Patent Application Publication No. 2004/0171154; Storici et al., 2001, Nature Biotechnol. 19: 773-776; Kren et al., 1998, Nat. Med. 4: 285-290; and Calissano and Macino, 1996, Fungal Genet. Newslett. 43: 15-16.

Any site-directed mutagenesis procedure can be used in the present invention. There are many commercial kits available that can be used to prepare variants.

Synthetic gene construction entails in vitro synthesis of a designed polynucleotide molecule to encode a polypeptide of interest. Gene synthesis can be performed utilizing a number of techniques, such as the multiplex microchip-based technology described by Tian et al. (2004, Nature 432: 1050-1054) and similar technologies wherein oligonucleotides are synthesized and assembled upon photo-programmable microfluidic chips.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204) and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled.

### Polynucleotides

The present invention also relates to isolated polynucleotides encoding a polypeptide having carbohydrate binding activity, as described herein.

The techniques used to isolate or clone a polynucleotide are known in the art and include isolation from genomic DNA or cDNA, or a combination thereof. The cloning of the polynucleotides from genomic DNA can be effected, e.g., by using the polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used.

Modification of a polynucleotide encoding a polypeptide of the present invention may be necessary for synthesizing polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that differ in specific activity, thermostability, pH optimum, or the like. The variants may be constructed on the basis of the polynucleotide presented as the mature polypeptide coding sequence of SEQ ID NO: e.g., a subsequence thereof, and/or by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2: 95-107.

### Nucleic Acid Constructs

The present invention also relates to nucleic acid constructs comprising a polynucleotide of the present invention, wherein the polynucleotide is operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the polynucleotide of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the polynucleotide of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase I, Trichoderma reesei endoglucanase V, Trichoderma reesei xylanase I, Trichoderma reesei xylanase II, Trichoderma reesei xylanase Ill, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase Ill, Trichoderma reesei endoglucanase V, Trichoderma reesei xylanase I, Trichoderma reesei xylanase II, Trichoderma reesei xylanase Ill, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, J. Bacteriol. 177: 3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is heterologous to the coding sequence. A heterologous signal peptide coding sequence may be required where the coding sequence does not naturally contain a signal peptide coding sequence. Alternatively, a heterologous signal peptide coding sequence may simply replace the natural signal peptide coding sequence to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alphaamylase, Bacillus stearothermophilus neutral proteases (nprT, nprS, nprM), and Bacillus subtilis prsA. Further signal peptides are described by Simonen and Palva, 1993, Microbiol. Rev. 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucormiehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosylaminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is a hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell.

Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMB1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more control sequences that direct the production of a polypeptide of the present invention. A construct or vector comprising a polynucleotide is introduced into a host cell so that the construct or vector is maintained as a chromosomal integrant or as a self-replicating extra-chromosomal vector as described earlier.

The choice of a host cell will to a large extent depend upon the gene encoding the polypeptide and its source.

In some embodiments, the polypeptide is heterologous to the recombinant host cell.

In some embodiments, at least one of the one or more control sequences is heterologous to the polynucleotide encoding the polypeptide.

In some embodiments, the recombinant host cell comprises at least two copies, e.g., three, four, or five, of the polynucleotide of the present invention.

The host cell may be any microbial or plant cell useful in the recombinant production of a polypeptide of the present invention, e.g., a prokaryotic cell or a fungal cell.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a polypeptide of the present invention, comprising (a) cultivating a cell, which in its wild-type form produces the polypeptide, under conditions conducive for production of the polypeptide; and optionally, (b) recovering the polypeptide.

The present invention also relates to methods of producing a polypeptide of the present invention, comprising (a) cultivating a recombinant host cell of the present invention under conditions conducive for production of the polypeptide; and optionally, (b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid-state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide The polypeptide may be recovered using methods known in the art. For example, the polypeptide may be recovered from the fermentation medium by conventional procedures including, but not limited to, collection, centrifugation, filtration, extraction, spray-drying, evaporation, or precipitation. In one aspect, a whole fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

### Fermentation Broth Formulations or Cell Compositions

The present invention also relates to a fermentation broth formulation or a cell composition comprising a polypeptide of the present invention. The fermentation broth formulation or the cell composition further comprises additional ingredients used in the fermentation process, such as, for example, cells (including, the host cells containing the gene encoding the polypeptide of the present invention which are used to produce the polypeptide of interest), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In some embodiments, the fermentation broth formulation or the cell composition comprises a first organic acid component comprising at least one 1-5 carbon organic acid and/or a salt thereof and a second organic acid component comprising at least one 6 or more carbon organic acid and/or a salt thereof. In some embodiments, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In some embodiments, the killed cells and/or cell debris are removed from a cell-killed whole broth to provide a composition that is free of these components.

The fermentation broth formulation or cell composition may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis. In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell composition of the present invention may be produced by a method described in WO 90/15861 or WO 2010/096673.

### Detergent Compositions

In one embodiment, the invention is directed to detergent compositions comprising a polypeptide of the present invention in combination with one or more additional cleaning composition components. The choice of additional components is within the skill of the artisan and includes conventional ingredients, including the exemplary non-limiting components set forth below.

The choice of components may include, for textile care, the consideration of the type of textile to be cleaned, the type and/or degree of soiling, the temperature at which cleaning is to take place, and the formulation of the detergent product. Although components mentioned below are categorized by general header according to a particular functionality, this is not to be construed as a limitation, as a component may comprise additional functionalities as will be appreciated by the skilled artisan.

### Surfactants

The detergent composition may comprise one or more surfactants, which may be anionic and/or cationic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 0.1% to 60% by weight, such as about 1% to about 40%, or about 3% to about 20%, or about 3% to about 10%. The surfactant(s) is chosen based on the desired cleaning application, and may include any conventional surfactant(s) known in the art.

When included therein the detergent will usually contain from about 1% to about 40% by weight of an anionic surfactant, such as from about 5% to about 30%, including from about 5% to about 15%, or from about 15% to about 20%, or from about 20% to about 25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenyl-alkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ether sulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap), and combinations thereof.

When included therein the detergent will usually contain from about 1% to about 40% by weigh of a cationic surfactant, for example from about 0.5% to about 30%, in particular from about 1% to about 20%, from about 3% to about 10%, such as from about 3% to about 5%, from about 8% to about 12% or from about 10% to about 12%. Non-limiting examples of cationic surfactants include alkyldimethylethanolamine quat (ADMEAQ), cetyltrimethylammonium bromide (CTAB), dimethyl-distearylammonium chloride (DSDMAC), and alkylbenzyldimethylammonium, alkyl quaternary ammonium compounds, alkoxylated quaternary ammonium (AQA) compounds, ester quats, and combinations thereof.

When included therein the detergent will usually contain from about 0.2% to about 40% by weight of a nonionic surfactant, for example from about 0.5% to about 30%, in particular, from about 1% to about 20%, from about 3% to about 10%, such as from about 3% to about 5%, from about 8% to about 12%, or from about 10% to about 12%. Non-limiting examples of nonionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof.

When included therein the detergent will usually contain from about 0.2% to about 10% by weight of a semipolar surfactant. Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, and combinations thereof.

When included therein the detergent will usually contain from about 0.2% to about 10% by weight of a zwitterionic surfactant. Non-limiting examples of zwitterionic surfactants include betaines such as alkyldimethylbetaines, sulfobetaines, and combinations thereof.

### Hydrotropes

A hydrotrope is a compound that solubilises hydrophobic compounds in aqueous solutions (or oppositely, polar substances in a non-polar environment). Typically, hydrotropes have both hydrophilic and a hydrophobic character (so-called amphiphilic properties as known from surfactants); however, the molecular structure of hydrotropes generally do not favor spontaneous self-aggregation, see e.g. review by Hodgdon and Kaler (2007), Current Opinion in Colloid & Interface Science 12: 121-128. Hydrotropes do not display a critical concentration above which self-aggregation occurs as found for surfactants and lipids forming miceller, lamellar or other well defined meso-phases. Instead, many hydrotropes show a continuous-type aggregation process where the sizes of aggregates grow as concentration increases. However, many hydrotropes alter the phase behavior, stability, and colloidal properties of systems containing substances of polar and non-polar character, including mixtures of water, oil, surfactants, and polymers. Hydrotropes are classically used across industries from pharma, personal care, food, to technical applications. Use of hydrotropes in detergent compositions allow for example more concentrated formulations of surfactants (as in the process of compacting liquid detergents by removing water) without inducing undesired phenomena such as phase separation or high viscosity.

The detergent may contain 0-10% by weight, for example 0-5% by weight, such as about 0.5 to about 5%, or about 3% to about 5%, of a hydrotrope. Any hydrotrope known in the art for use in detergents may be utilized. Non-limiting examples of hydrotropes include sodium benzenesulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

### Builders and Co-Builders

The detergent composition may contain about 0-65% by weight, such as about 5% to about 50% of a detergent builder or co-builder, or a mixture thereof. In a dish wash detergent, the level of builder is typically 40-65%, particularly 50-65%. The builder and/or co-builder may particularly be a chelating agent that forms water-soluble complexes with Ca and Mg. Any builder and/or co-builder known in the art for use in laundry detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as 2,2′-iminodiethan-1-ol), triethanolamine (TEA, also known as 2,2′,2″-nitrilotriethan-1-ol), and (carboxymethyl)inulin (CMI), and combinations thereof.

The detergent composition may also contain 0-50% by weight, such as about 5% to about 30%, of a detergent co-builder. The detergent composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1, 1-diphosphonic acid (HEDP), ethylenediaminetetra(methylenephosphonicacid) (EDTMPA), diethylenetriaminepentakis(methylenephosphonicacid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N(2-sulfomethyl)-glutamic acid (SMGL), N-(2-sulfoethyl)-glutamic acid (SEGL), N-methyliminodiacetic acid (MIDA), a-alanine-N,N-diacetic acid (a-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilicacid-N,N-diacetic acid (SLDA), taurine-N,N-diacetic acid (TUDA) and sulfomethylN,N-diacetic acid (SMDA), N-(2-hydroxyethyl)ethylenediamine-N,N′,N′-triacetic acid (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 09/102854, U.S. Pat. No. 5,977,053

### Bleaching Systems

The detergent may contain 0-30% by weight, such as about 1% to about 20%, of a bleaching system. Any bleaching system known in the art for use in laundry detergents may be utilized. Suitable bleaching system components include bleaching catalysts, photobleaches, bleach activators, sources of hydrogen peroxide such as sodium percarbonate, sodium perborates and hydrogen peroxide-urea (1:1), preformed peracids and mixtures thereof. Suitable preformed peracids include, but are not limited to, peroxycarboxylic acids and salts, diperoxydicarboxylic acids, perimidic acids and salts, peroxymonosulfuric acids and salts, for example, Oxone®, and mixtures thereof. Non-limiting examples of bleaching systems include peroxide-based bleaching systems, which may comprise, for example, an inorganic salt, including alkali metal salts such as sodium salts of perborate (usually mono- or tetra-hydrate), percarbonate, persulfate, perphosphate, persilicate salts, in combination with a peracid-forming bleach activator. The term bleach activator is meant herein as a compound which reacts with hydrogen peroxide to form a peracid via perhydrolysis. The peracid thus formed constitutes the activated bleach. Suitable bleach activators to be used herein include those belonging to the class of esters, amides, imides or anhydrides. Suitable examples are tetraacetylethylenediamine (TAED), sodium 4-[(3,5,5-trimethylhexanoyl)oxy]benzene-1-sulfonate (ISONOBS), 4-(dodecanoyloxy)benzene-1-sulfonate (LOBS), 4-(decanoyloxy)benzene-1-sulfonate, 4-(decanoyloxy)benzoate (DOBS or DOBA), 4-(nonanoyloxy)benzene-1-sulfonate (NOBS), and/or those disclosed in WO98/17767. A particular family of bleach activators of interest was disclosed in EP624154 and particularly preferred in that family is acetyl triethyl citrate (ATC). ATC or a short chain triglyceride like triacetin has the advantage that it is environmentally friendly Furthermore acetyl triethyl citrate and triacetin have good hydrolytical stability in the product upon storage and are efficient bleach activators. Finally ATC is multifunctional, as the citrate released in the perhydrolysis reaction may function as a builder. Alternatively, the bleaching system may comprise peroxyacids of, for example, the amide, imide, or sulfone type. The bleaching system may also comprise peracids such as 6-(phthalimido)peroxyhexanoic acid (PAP). The bleaching system may also include a bleach catalyst. In some embodiments the bleach component may be an organic catalyst selected from the group consisting of organic catalysts having the following formulae:

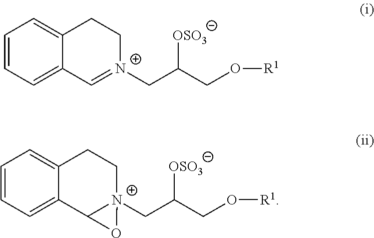

(iii) and mixtures thereof;

wherein each R¹ is independently a branched alkyl group containing from 9 to 24 carbons or linear alkyl group containing from 11 to 24 carbons, preferably each R¹ is independently a branched alkyl group containing from 9 to 18 carbons or linear alkyl group containing from 11 to 18 carbons, more preferably each R¹ is independently selected from the group consisting of 2-propylheptyl, 2-butyloctyl, 2-pentylnonyl, 2-hexyldecyl, dodecyl, tetradecyl, hexadecyl, octadecyl, isononyl, isodecyl, isotridecyl and isopentadecyl. Other exemplary bleaching systems are described, e.g. in WO2007/087258, WO2007/087244, WO2007/087259, EP1867708 (Vitamin K) and WO2007/087242. Suitable photobleaches may for example be sulfonated zinc or aluminium phthalocyanines.

Preferably the bleach component comprises a source of peracid in addition to bleach catalyst, particularly organic bleach catalyst. The source of peracid may be selected from (a) preformed peracid; (b) percarbonate, perborate or persulfate salt (hydrogen peroxide source) preferably in combination with a bleach activator; and (c) perhydrolase enzyme and an ester for forming peracid in situ in the presence of water in a textile or hard surface treatment step.

### Polymers

The detergent may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidonevinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated.

### Fabric Hueing Agents

The detergent compositions of the present invention may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO2005/03274, WO2005/03275, WO2005/03276 and EP1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch. Suitable hueing agents are also disclosed in, e.g. WO 2007/087257 and WO2007/087243.

### Enzymes

The detergent additive as well as the detergent composition may comprise one or more enzymes such as a protease, lipase, cutinase, an amylase, carbohydrase, cellulase, pectinase, mannanase, arabinase, galactanase, xylanase, nuclease, oxidase, e.g., a laccase, and/or peroxidase.

In general, the properties of the selected enzyme(s) should be compatible with the selected detergent, (i.e., pH-optimum, compatibility with other enzymatic and non-enzymatic ingredients, etc.), and the enzyme(s) should be present in effective amounts.

Cellulases

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. Nos. 4,435,307, 5,648,263, 5,691,178, 5,776,757 and WO 89/09259.

Especially suitable cellulases are the alkaline or neutral cellulases having colour care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. Nos. 5,457,046, 5,686,593, 5,763,254, WO 95/24471, WO 98/12307 and WO99/001544.

Other cellulases are endo-beta-1,4-glucanase enzyme having a sequence of at least 97% identity to the amino acid sequence of position 1 to position 773 of SEQ ID NO:2 of WO 2002/099091 or a family 44 xyloglucanase, which a xyloglucanase enzyme having a sequence of at least 60% identity to positions 40-559 of SEQ ID NO: 2 of WO 2001/062903.

Commercially available cellulases include Celluzyme™, and Carezyme™ (Novozymes A/S) Carezyme Premium™ (Novozymes A/S), Celluclean™ (Novozymes A/S), Celluclean Classic™ (Novozymes A/S), Cellusoft™ (Novozymes A/S), Whitezyme™ (Novozymes A/S), Clazinase™, and Puradax HA™ (Genencor International Inc.), and KAC-500(B)™ (Kao Corporation).

Mannanases

Suitable mannanases include those of bacterial or fungal origin. Chemically or genetically modified mutants are included. The mannanase may be an alkaline mannanase of Family 5 or 26. It may be a wild-type from Bacillus or Humicola, particularly B. agaradhaerens, B. licheniformis, B. halodurans, B. clausii, or H. insolens. Suitable mannanases are described in WO 1999/064619. A commercially available mannanase is Mannaway (Novozymes A/S).

### Cellulase

Suitable cellulases include complete cellulases or mono-component endoglucanases of bacterial or fungal origin. Chemically or genetically modified mutants are included. The cellulase may for example be a mono-component or a mixture of mono-component endo-1,4-beta-glucanase often just termed endoglucanases. Suitable cellulases include a fungal cellulase from Humicola insolens (U.S. Pat. No. 4,435,307) or from Trichoderma, e.g. T. reesei or T. viride. Examples of cellulases are described in EP 0 495 257. Other suitable cellulases are from Thielavia e.g. Thielavia terrestris as described in WO 96/29397 or Fusarium oxysporum as described in WO 91/17244 or from Bacillus as described in, WO 02/099091 and JP 2000210081. Other examples are cellulase variants such as those described in WO 94/07998, EP 0 531 315, U.S. Pat. Nos. 5,457,046, 5,686,593, 5,763,254, WO 95/24471, WO 98/12307 Commercially available cellulases include Carezyme®, Celluzyme®, Celluclean®, Celluclast® and Endolase®; Renozyme®; Whitezyme® (Novozymes A/S) Puradax®, Puradax HA, and Puradax EG (available from Genencor).

### Peroxidases/Oxidases

Suitable peroxidases/oxidases include those of plant, bacterial or fungal origin.

Chemically modified or protein engineered mutants are included. Examples of useful peroxidases include peroxidases from Coprinus, e.g., from C. cinereus, and variants thereof as those described in WO 93/24618, WO 95/10602, and WO 98/15257. Commercially available peroxidases include Guardzyme™ (Novozymes A/S).

### Proteases

Suitable proteases include those of bacterial, fungal, plant, viral or animal origin e.g. vegetable or microbial origin. Microbial origin is preferred. Chemically modified or protein engineered mutants are included. It may be an alkaline protease, such as a serine protease or a metalloprotease. A serine protease may for example be of the S1 family, such as trypsin, or the S8 family such as subtilisin. A metalloproteases protease may for example be a thermolysin from e.g. family M4 or other metalloprotease such as those from M5, M7 or M8 families.

The term “subtilases” refers to a sub-group of serine protease according to Siezen et al., Protein Engng. 4 (1991) 719-737 and Siezen et al. Protein Science 6 (1997) 501-523. Serine proteases are a subgroup of proteases characterized by having a serine in the active site, which forms a covalent adduct with the substrate. The subtilases may be divided into 6 sub-divisions, i.e. the Subtilisin family, the Thermitase family, the Proteinase K family, the Lantibiotic peptidase family, the Kexin family and the Pyrolysin family.

Examples of subtilases are those derived from Bacillus such as Bacillus lentus, Bacillus alkalophilus, Bacillus subtilis, Bacillus amyloliquefaciens, Bacillus pumilus and Bacillus gibsonii described in; U.S. Pat. No. 7,262,042 and WO09/021867, and Subtilisin lentus, Subtilisin Novo, subtilisin Carlsberg, Bacillus licheniformis, subtilisin BPN′, subtilisin 309, subtilisin 147 and subtilisin 168 and e.g. protease PD138 described in (WO93/18140). Other useful proteases may be those described in WO01/016285 and WO02/016547. Examples of trypsin-like proteases are trypsin (e.g. of porcine or bovine origin) and the Fusarium protease described in WO94/25583 and WO05/040372, and the chymotrypsin proteases derived from Cellumonas described in WO05/052161 and WO05/052146.

A further preferred protease is the alkaline protease from Bacillus lentus DSM 5483, as described for example in WO95/23221, and variants thereof which are described in WO92/21760, WO95/23221, EP1921147 and EP1921148.

Examples of metalloproteases are the neutral metalloprotease as described in WO07/044993 (Proctor & Gamble/Genencor Int.) such as those derived from Bacillus amyloliquefaciens.

Examples of useful proteases are the variants described in: WO89/06279 WO92/19729, WO96/034946, WO98/20115, WO98/20116, WO99/011768, WO01/44452, WO03/006602, WO04/03186, WO04/041979, WO07/006305, WO11/036263, WO11/036264, especially the variants with substitutions in one or more of the following positions: 3, 4, 9, 15, 24, 27, 42, 55, 59, 60, 66, 74, 85, 96, 97, 98, 99, 100, 101, 102, 104, 116, 118, 121, 126, 127, 128, 154, 156, 157, 158, 161, 164, 176, 179, 182, 185, 188, 189, 193, 198, 199, 200, 203, 206, 211, 212, 216, 218, 226, 229, 230, 239, 246, 255, 256, 268 and 269 wherein the positions correspond to the positions of the Bacillus lentus protease shown in SEQ ID NO 1 of WO 2016/001449. More preferred the protease variants may comprise one or more of the mutations selected from the group consisting of: S3T, V4I, S9R, S9E, A15T, S24G, S24R, K27R, N42R, S55P, G59E, G59D, N60D, N60E, V66A, N74D, S85R, A96S, S97G, S97D, S97A, S97SD, S99E, S99D, S99G, S99M, S99N, S99R, S99H, S101A, V102I, V102Y, V102N, S104A, G116V, G116R, H118D, H118N, A120S, S126L, P127Q, S128A, S154D, A156E, G157D, G157P, S158E, Y161A, R164S, Q176E, N179E, S182E, Q185N, A188P, G189E, V193M, N198D, V1991, Y203W, S206G, L211Q, L211D, N212D, N212S, M216S, A226V, K229L, Q230H, Q239R, N246K, N255W, N255D, N255E, L256E, L256D T268A and R269H. The protease variants are preferably variants of the Bacillus lentus protease (Savinase®) shown in SEQ ID NO 1 of WO2016/001449, the Bacillus amylolichenifaciens protease (BPN′) shown in SEQ ID NO 2 of WO2016/001449. The protease variants preferably have at least 80% sequence identity to SEQ ID NO 1 or SEQ ID NO 2 of WO 2016/001449.

A protease variant comprising a substitution at one or more positions corresponding to positions 171, 173, 175, 179, or 180 of SEQ ID NO: 1 of WO2004/067737, wherein said protease variant has a sequence identity of at least 75% but less than 100% to SEQ ID NO: 1 of WO2004/067737.

Suitable commercially available protease enzymes include those sold under the trade names Alcalase®, Duralase™, Durazym™, Relase®, Relase® Ultra, Savinase®, Savinase® Ultra, Primase®, Polarzyme®, Kannase®, Liquanase®, Liquanase® Ultra, Novozymes Progress®, Novozymes Progress® Uno, Novozymes Progress® Excell, Ovozyme®, Coronase®, Coronase® Ultra, Blaze®, Blaze Evity® 100T, Blaze Evity® 125T, Blaze Evity® 150T, Neutrase®, Everlase® and Esperase® (Novozymes A/S), those sold under the tradename Maxatase®, Maxacal®, Maxapem®, Purafect Ox®, Purafect OxP®, Puramax®, FN2®, FN3®, FN4®, Excellase®, Excellenz P110™, Excellenz P1000™, Eraser®, Preferenz P100™, Purafect Prime®, Preferenz P110™, Effectenz P1000™, Purafect®™, Effectenz P1050™, Purafect Ox®™, Effectenz P2000™, Purafast®, Properase®, Opticlean® and Optimase® (Danisco/DuPont), Axapem™ (Gist-Brocases N.V.), BLAP (sequence shown in FIG. 29 of U.S. Pat. No. 5,352,604) and variants hereof (Henkel AG) and KAP (Bacillus alkalophilus subtilisin) from Kao.

Lipases and Cutinases:

Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include include Lipolase™, Lipex™; Lipolex™ and Lipoclean™ (Novozymes A/S), Lumafast (originally from Genencor) and Lipomax (originally from Gist-Brocades).

Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

Amylases:

Suitable amylases which can be used together with the polypeptides of the invention may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

M197T;

H156Y+A181T+N190F+A209V+Q264S; or

G48A+T491+G107A+H156Y+A181T+N190F+1201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, 1206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO 96/023873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T1651, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

N128C+K178L+T182G+Y305R+G475K;

N128C+K178L+T182G+F202Y+Y305R+D319T+G475K;

S125A+N128C+K178L+T182G+Y305R+G475K; or

S125A+N128C+T1311+T165+K178L+T182G+Y305R+G475K wherein the variants are C-terminally truncated and optionally further comprises a substitution at position 243 and/or a deletion at position 180 and/or position 181.

Further suitable amylases are amylases having SEQ ID NO: 1 of WO13184577 or variants having 90% sequence identity to SEQ ID NO: 1 thereof. Preferred variants of SEQ ID NO: 1 are those having a substitution, a deletion or an insertion in one of more of the following positions: K176, R178, G179, T180, G181, E187, N192, M199, I203, S241, R458, T459, D460, G476 and G477. More preferred variants of SEQ ID NO: 1 are those having the substitution in one of more of the following positions: K176L, E187P, N192FYH, M199L, I203YF, S241QADN, R458N, T459S, D460T, G476K and G477K and/or deletion in position R178 and/or S179 or of T180 and/or G181. Most preferred amylase variants of SEQ ID NO: 1 are those having the substitutions:

E187P+1203Y+G476K

E187P+1203Y+R458N+T459S+D460T+G476K

wherein the variants optionally further comprise a substitution at position 241 and/or a deletion at position 178 and/or position 179.

Further suitable amylases are amylases having SEQ ID NO: 1 of WO10104675 or variants having 90% sequence identity to SEQ ID NO: 1 thereof. Preferred variants of SEQ ID NO: 1 are those having a substitution, a deletion or an insertion in one of more of the following positions: N21, D97, V128 K177, R179, S180, I181, G182, M200, L204, E242, G477 and G478. More preferred variants of SEQ ID NO: 1 are those having the substitution in one of more of the following positions: N21 D, D97N, V128I K177L, M200L, L204YF, E242QA, G477K and G478K and/or deletion in position R179 and/or S180 or of I181 and/or G182. Most preferred amylase variants of SEQ ID NO: 1 are those having the substitutions:

N21D+D97N+V128I

wherein the variants optionally further comprise a substitution at position 200 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Duramyl™, Termamyl™, Fungamyl™, Stainzyme™, Stainzyme Plus™, Natalase™, Liquozyme X and BAN™ (from Novozymes A/S), and Rapidase™, Purastar™/Effectenz™, Powerase, Preferenz S1000, Preferenz S100 and Preferenz S110 (from Genencor International Inc./DuPont).

Peroxidases/Oxidases

A peroxidase according to the invention is a peroxidase enzyme comprised by the enzyme classification EC 1.11.1.7, as set out by the Nomenclature Committee of the International Union of Biochemistry and Molecular Biology (IUBMB), or any fragment derived therefrom, exhibiting peroxidase activity.

Suitable peroxidases include those of plant, bacterial or fungal origin. Chemically modified or protein engineered mutants are included. Examples of useful peroxidases include peroxidases from Coprinopsis, e.g., from C. cinerea (EP 179,486), and variants thereof as those described in WO 93/24618, WO 95/10602, and WO 98/15257.

A peroxidase according to the invention also include a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions.

In an embodiment, the haloperoxidase of the invention is a chloroperoxidase. Preferably, the haloperoxidase is a vanadium haloperoxidase, i.e., a vanadate-containing haloperoxidase. In a preferred method of the present invention the vanadate-containing haloperoxidase is combined with a source of chloride ion.

Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis.

Haloperoxidases have also been isolated from bacteria such as Pseudomonas, e.g., P. pyrrocinia and Streptomyces, e.g., S. aureofaciens.

In an preferred embodiment, the haloperoxidase is derivable from Curvularia sp., in particular Curvularia verruculosa or Curvularia inaequalis, such as C. inaequalis CBS 102.42 as described in WO 95/27046; or C. verruculosa CBS 147.63 or C. verruculosa CBS 444.70 as described in WO 97/04102; or from Drechslera hartlebii as described in WO 01/79459, Dendryphiella salina as described in WO 01/79458, Phaeotrichoconis crotalarie as described in WO 01/79461, or Geniculosporium sp. as described in WO 01/79460.

An oxidase according to the invention include, in particular, any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5).

Preferred laccase enzymes are enzymes of microbial origin. The enzymes may be derived from plants, bacteria or fungi (including filamentous fungi and yeasts).

Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radiata (WO 92/01046), or Coriolus, e.g., C. hirsutus (JP 2238885).

Suitable examples from bacteria include a laccase derivable from a strain of Bacillus.

A laccase derived from Coprinopsis or Myceliophthora is preferred; in particular a laccase derived from Coprinopsis cinerea, as disclosed in WO 97/08325; or from Myceliophthora thermophila, as disclosed in WO 95/33836.

Nucleases

Suitable nucleases include deoxyribonucleases (DNases) as well as ribonucleases. DNases are any enzyme that catalyzes the hydrolytic cleavage of phosphodiester linkages in the DNA backbone, thus degrading DNA. According to the invention, a DNase which is obtainable from a bacterium is preferred; in particular a DNase, which is obtainable from a Bacillus is preferred; in particular a DNase which is obtainable from Bacillus subtilis or Bacillus licheniformis is preferred. Examples of such DNases are described in patent application WO 2011/098579 or in PCT/EP2013/075922.

The detergent enzyme(s) may be included in a detergent composition by adding separate additives containing one or more enzymes, or by adding a combined additive comprising all of these enzymes. A detergent additive of the invention, i.e., a separate additive or a combined additive, can be formulated, for example, as a granulate, liquid, slurry, etc. Preferred detergent additive formulations are granulates, in particular non-dusting granulates, liquids, in particular stabilized liquids, or slurries.

Non-dusting granulates may be produced, e.g. as disclosed in U.S. Pat. Nos. 4,106,991 and 4,661,452 and may optionally be coated by methods known in the art. Examples of waxy coating materials are polyethyleneglycol (PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238,216.

### Microorganisms

The detergent additive as well as the detergent composition may also comprise one or more microorganisms, such as one or more fungi, yeast, or bacteria.

In an embodiment, the one or more microorganisms are dehydrated (for example by lyophilization) bacteria or yeast, such as a strain of Lactobacillus.

In another embodiment, the microorganisms are one or more microbial spores (as opposed to vegetative cells), such as bacterial spores; or fungal spores, conidia, hypha. Preferably, the one or more spores are Bacillus endospores; even more preferably the one or more spores are endospores of Bacillus subtilis, Bacillus licheniformis, Bacillus amyloliquefaciens, or Bacillus megaterium.

The microorganisms may be included in the detergent composition or additive in the same way as enzymes (see above).

### Adjunct Materials

Any detergent components known in the art for use in laundry detergents may also be utilized. Other optional detergent components include anti-corrosion agents, additional anti-shrink agents, anti-soil redeposition agents, anti-wrinkling agents, bactericides, binders, corrosion inhibitors, disintegrants/disintegration agents, dyes, enzyme stabilizers (including boric acid, borates, CMC, and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, tarnish inhibitors, and wicking agents, either alone or in combination. Any ingredient known in the art for use in laundry detergents may be utilized. The choice of such ingredients is well within the skill of the artisan.

### Dispersants

The detergent compositions of the present invention can also contain dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

### Dye Transfer Inhibiting Agents

The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof.

When present in a subject composition, the dye transfer inhibiting agents may be present at levels from about 0.0001% to about 10%, from about 0.01% to about 5% or even from about 0.1% to about 3% by weight of the composition.

### Fluorescent Whitening Agent

The detergent compositions of the present invention will preferably also contain additional components that may tint articles being cleaned, such as fluorescent whitening agent or optical brighteners. Where present the brightener is preferably at a level of about 0.01% to about 0.5%. Any fluorescent whitening agent suitable for use in a laundry detergent composition may be used in the composition of the present invention. The most commonly used fluorescent whitening agents are those belonging to the classes of diaminostilbene-sulfonic acid derivatives, diarylpyrazoline derivatives and bisphenyl-distyryl derivatives. Examples of the diaminostilbene-sulfonic acid derivative type of fluorescent whitening agents include the sodium salts of: 4,4′-bis-(2-diethanolamino-4-anilino-s-triazin-6-ylamino) stilbene-2,2′-disulfonate, 4,4′-bis-(2,4-dianilino-s-triazin-6-ylamino) stilbene-2,2′-disulfonate, 4,4′-bis-(2-anilino-4-(N-methyl-N-2-hydroxy-ethylamino)-s-triazin-6-ylamino) stilbene-2,2′-disulfonate, 4,4′-bis-(4-phenyl-1,2,3-triazol-2-yl)stilbene-2,2′-disulfonate and sodium 5-(2H-naphtho[1,2-d][1,2,3]triazol-2-yl)-2-[(E)-2-phenylvinyl]benzenesulfonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4-anilino-s-triazin-6-ylamino) stilbene-2,2′-disulfonate. Tinopal CBS is the disodium salt of 2,2′-bis-(phenyl-styryl)-disulfonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins.

Suitable fluorescent brightener levels include lower levels of from about 0.01, from 0.05, from about 0.1 or even from about 0.2 wt % to upper levels of 0.5 or even 0.75 wt %.

### Soil Release Polymers

The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore random graft co-polymers are suitable soil release polymers. Suitable graft co-polymers are described in more detail in WO 2007/138054, WO 2006/108856 and WO 2006/113314 (hereby incorporated by reference). Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

### Anti-Redeposition Agents

The detergent compositions of the present invention may also include one or more antiredeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as antiredeposition agents.

### Rheology Modifiers

The detergent compositions of the present invention may also include one or more rheology modifiers, structurants or thickeners, as distinct from viscosity reducing agents. The rheology modifiers are selected from the group consisting of non-polymeric crystalline, hydroxy-functional materials, polymeric rheology modifiers which impart shear thinning characteristics to the aqueous liquid matrix of a liquid detergent composition. The rheology and viscosity of the detergent can be modified and adjusted by methods known in the art, for example as shown in EP 2169040.

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

### Formulation of Detergent Products

The detergent composition of the invention may be in any convenient form, e.g., a bar, a homogenous tablet, a tablet having two or more layers, a pouch having one or more compartments, a regular or compact powder, a granule, a paste, a gel, or a regular, compact or concentrated liquid.

Pouches can be configured as single or multicompartments. It can be of any form, shape and material which is suitable for hold the composition, e.g. without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid laundry cleaning composition or part components and/or a liquid cleaning composition or part components separated by the water soluble film. The compartment for liquid components can be different in composition than compartments containing solids: US2009/0011970 A1.

Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent, which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent.

A liquid or gel detergent may be non-aqueous.

### Laundry Soap Bars

The polypeptides of the invention may be added to laundry soap bars and used for hand washing laundry, fabrics and/or textiles. The term laundry soap bar includes laundry bars, soap bars, combo bars, syndet bars and detergent bars. The types of bar usually differ in the type of surfactant they contain, and the term laundry soap bar includes those containing soaps from fatty acids and/or synthetic soaps. The laundry soap bar has a physical form which is solid and not a liquid, gel or a powder at room temperature. The term solid is defined as a physical form which does not significantly change over time, i.e. if a solid object (e.g. laundry soap bar) is placed inside a container, the solid object does not change to fill the container it is placed in. The bar is a solid typically in bar form but can be in other solid shapes such as round or oval.

The laundry soap bar may contain one or more additional enzymes, protease inhibitors such as peptide aldehydes (or hydrosulfite adduct or hemiacetal adduct), boric acid, borate, borax and/or phenylboronic acid derivatives such as 4-formylphenylboronic acid, one or more soaps or synthetic surfactants, polyols such as glycerine, pH controlling compounds such as fatty acids, citric acid, acetic acid and/or formic acid, and/or a salt of a monovalent cation and an organic anion wherein the monovalent cation may be for example Na⁺, K⁺ or NH₄⁺ and the organic anion may be for example formate, acetate, citrate or lactate such that the salt of a monovalent cation and an organic anion may be, for example, sodium formate.

The laundry soap bar may also contain complexing agents like EDTA and HEDP, perfumes and/or different type of fillers, surfactants e.g. anionic synthetic surfactants, builders, polymeric soil release agents, detergent chelators, stabilizing agents, fillers, dyes, colorants, dye transfer inhibitors, alkoxylated polycarbonates, suds suppressers, structurants, binders, leaching agents, bleaching activators, clay soil removal agents, anti-redeposition agents, polymeric dispersing agents, brighteners, fabric softeners, perfumes and/or other compounds known in the art.

The laundry soap bar may be processed in conventional laundry soap bar making equipment such as but not limited to: mixers, plodders, e.g. a two stage vacuum plodder, extruders, cutters, logo-stampers, cooling tunnels and wrappers. The invention is not limited to preparing the laundry soap bars by any single method. The premix of the invention may be added to the soap at different stages of the process. For example, the premix containing a soap, polypeptide of the invention optionally one or more additional enzymes, a protease inhibitor, and a salt of a monovalent cation and an organic anion may be prepared and the mixture is then plodded. The polypeptides of the invention and optional additional enzymes may be added at the same time as the protease inhibitor for example in liquid form. Besides the mixing step and the plodding step, the process may further comprise the steps of milling, extruding, cutting, stamping, cooling and/or wrapping.

### Granular Detergent Formulations

A granular detergent may be formulated as described in WO09/092699, EP1705241, EP1382668, WO07/001262, U.S. Pat. No. 6,472,364, WO04/074419 or WO09/102854. Other useful detergent formulations are described in WO09/124162, WO09/124163, WO09/117340, WO09/117341, WO09/117342, WO09/072069, WO09/063355, WO09/132870, WO09/121757, WO09/112296, WO09/112298, WO09/103822, WO09/087033, WO09/050026, WO09/047125, WO09/047126, WO09/047127, WO09/047128, WO09/021784, WO09/010375, WO09/000605, WO09/122125, WO09/095645, WO09/040544, WO09/040545, WO09/024780, WO09/004295, WO09/004294, WO09/121725, WO09/115391, WO09/115392, WO09/074398, WO09/074403, WO09/068501, WO09/065770, WO09/021813, WO09/030632, and WO09/015951.

WO2011025615, WO2011016958, WO2011005803, WO2011005623, WO2011005730, WO2011005844, WO2011005904, WO2011005630, WO2011005830, WO2011005912, WO2011005905, WO2011005910, WO2011005813, WO2010135238, WO2010120863, WO2010108002, WO2010111365, WO2010108000, WO2010107635, WO2010090915, WO2010033976, WO2010033746, WO2010033747, WO2010033897, WO2010033979, WO2010030540, WO2010030541, WO2010030539, WO2010024467, WO2010024469, WO2010024470, WO2010025161, WO2010014395, WO2010044905,

WO2010145887, WO2010142503, WO2010122051, WO2010102861, WO2010099997, WO2010084039, WO2010076292, WO2010069742, WO2010069718, WO2010069957, WO2010057784, WO2010054986, WO2010018043, WO2010003783, WO2010003792,

WO2011023716, WO2010142539, WO2010118959, WO2010115813, WO2010105942, WO2010105961, WO2010105962, WO2010094356, WO2010084203, WO2010078979, WO2010072456, WO2010069905, WO2010076165, WO2010072603, WO2010066486, WO2010066631, WO2010066632, WO2010063689, WO2010060821, WO2010049187, WO2010031607, WO2010000636.

### Formulation of Enzyme in Co-Granule

The enzyme of the invention may be formulated as a granule for example as a co-granule that combines one or more enzymes. Each enzyme will then be present in more granules securing a more uniform distribution of enzymes in the detergent. This also reduces the physical segregation of different enzymes due to different particle sizes. Methods for producing multi-enzyme co-granulates for the detergent industry are disclosed in the IP .com disclosure IPCOM000200739D.

Another example of formulation of enzymes by the use of co-granulates are disclosed in WO 2013/188331, which relates to a detergent composition comprising (a) a multi-enzyme co-granule; (b) less than 10 wt zeolite (anhydrous basis); and (c) less than 10 wt phosphate salt (anhydrous basis), wherein said enzyme co-granule comprises from 10 to 98 wt % moisture sink component and the composition additionally comprises from 20 to 80 wt % detergent moisture sink component.

WO 2013/188331 also relates to a method of treating and/or cleaning a surface, preferably a fabric surface comprising the steps of (i) contacting said surface with the detergent composition as claimed and described herein in an aqueous wash liquor, (ii) rinsing and/or drying the surface.

The multi-enzyme co-granule may comprise an enzyme of the invention and (a) one or more enzymes selected from the group consisting of first-wash lipases, cleaning cellulases, xyloglucanases, perhydrolases, peroxidases, lipoxygenases, laccases and mixtures thereof; and (b) one or more enzymes selected from the group consisting of hemicellulases, proteases, care cellulases, cellobiose dehydrogenases, xylanases, phospho lipases, esterases, cutinases, pectinases, mannanases, pectate lyases, keratinases, reductases, oxidases, phenoloxidases, ligninases, pullulanases, lanases, pentosanases, lichenases glucanases, arabinosidases, hyaluronidase, chondroitinase, amylases, nucleases, and mixtures thereof. In another embodiment, the multi-enzyme co-granule does not comprise a cellulase.

### Use in Detergents.

The polypeptides of the present invention may be added to and thus become a component of a detergent composition.

The detergent composition of the present invention may be formulated, for example, as a hand or machine laundry detergent composition including a laundry additive composition suitable for pre-treatment of stained fabrics and a rinse added fabric softener composition, or be formulated as a detergent composition for use in general household hard surface cleaning operations, or be formulated for hand or machine dishwashing operations.

In a specific aspect, the present invention provides a detergent additive comprising a polypeptide of the present invention as described herein.

### PREFERRED EMBODIMENTS

Embodiment 1. A fusion polypeptide comprising at least two carbohydrate binding modules (CBMs) or fragments thereof, wherein the polypeptide has carbohydrate binding activity.
Embodiment 2. The polypeptide of embodiment 1, which is a non-naturally occurring multimer comprising at least two carbohydrate binding modules or fragments thereof.
Embodiment 3. The polypeptide of any preceding embodiment, comprising three or more CBMs, such as four or more CBMs, five or more CBMs, six or more CBMs, seven or more CBMs, eight of more CBMs, nine or more CBMs, ten or more CBMs, 11 or more CBMs, 12 or more CBMs, 13 or more CBMs, 14 or more CBMs, 15 or more CBMs, 16 or more CBMs, 17 or more CBMs, 18 or more CBMs, 19 or more CBMs, or even 20 CBMs.
Embodiment 4. The polypeptide of any preceding embodiment, wherein the at least two CBMs are the same or different and are each independently selected.
Embodiment 5. The polypeptide of any preceding embodiment, which is a heteromultimer.
Embodiment 6. The polypeptide of any preceding embodiment, wherein each CBM is independently selected among CBM family 1, 4, 17, 28, 30, 44, 72 and 79, and mixtures thereof; preferably wherein each CBM is a CBM family 1 CBM.
Embodiment 7. The polypeptide of any preceding embodiment, which is a comprising three, four, or five CBMs, each from CBM Family 1; preferably comprising three different CBMs, each from CBM Family 1.
Embodiment 8. The polypeptide of any preceding embodiment, wherein the CBMs are joined by a linker region.
Embodiment 9. The polypeptide of any preceding embodiment, wherein the linker region is heterologous to each of the CBMs.
Embodiment 10. The polypeptide of any preceding embodiment, wherein each CBM is independently selected among polypeptides having at least 60% sequence identity to one of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 23 e.g. at least 70%, sequence identity, e.g. at least 80% sequence identity, e.g. at least 90% sequence identity; e.g. at least 95%, sequence identity, e.g. at least 96% sequence identity, e.g. at least 97% sequence identity; e.g. at least 98% sequence identity or at least 99% sequence identity, or even 100% sequence identity.
Embodiment 11. The polypeptide of any preceding embodiment, wherein each CBM is independently selected from a CBM having the amino acid sequence of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22 or having an amino acid sequence that deviates from one of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 23 by 1, 2, 3, 4, 5, 6, 7, 8 or 9 substitutions, insertions or deletions.
Embodiment 12. The polypeptide of any preceding embodiment, comprising four CBMs having the amino acid sequence of SEQ ID NO: 2, SEQ ID NO: 4, SEQ ID NO: 6 and SEQ ID NO: 23, or having an amino acid sequence that deviates from one of SEQ ID NO: 2, SEQ ID NO: 4, SEQ ID NO: 6 and SEQ ID NO: 23 by 1, 2, 3, 4, 5, 6, 7, 8 or 9 substitutions, insertions or deletions.
Embodiment 13. The polypeptide of any preceding embodiment, having at least 60% sequence identity, e.g., 70% sequence identity, e.g. at least 80% sequence identity, e.g. at least 90% sequence identity; e.g. at least 95%, sequence identity, e.g. at least 96% sequence identity, e.g. at least 97% sequence identity; e.g. at least 98% sequence identity or at least 99% sequence identity, or even 100% sequence identity to the polypeptide of SEQ ID NO: 30, SEQ ID NO: 32, or SEQ ID NO: 38.
Embodiment 14. The polypeptide of any preceding embodiment, comprising a CBM derived from a fungus.
Embodiment 15. Use of the fusion polypeptide of any of embodiments 1-14 for reducing wrinkles and/or providing increased anti-crease properties and/or providing improved ease of ironing and/or providing improved shape retention in a cleaning process of a fabric or textile.
Embodiment 16. The use of embodiment 15, wherein the fabric or textile is contacted with a liquid solution comprising a polypeptide having carbohydrate binding activity.
Embodiment 17. The use of any of embodiments 15-16, wherein the liquid solution is a wash liquor.
Embodiment 18. The use of any of the preceding use embodiments, provided as a laundry booster.
Embodiment 19. The use of any of the preceding use embodiments, wherein the polypeptide is a non-naturally occurring multimer comprising at least two carbohydrate binding modules.
Embodiment 20. The use of any of the preceding use embodiments, wherein the polypeptide comprises three or more CBMs, such as four or more CBMs, five or more CBMs, six or more CBMs, seven or more CBMs, eight of more CBMs, nine or more CBMs, ten or more CBMs, 11 or more CBMs, 12 or more CBMs, 13 or more CBMs, 14 or more CBMs, 15 or more CBMs, 16 or more CBMs, 17 or more CBMs, 18 or more CBMs, 19 or more CBMs, or even 20 CBMs.
Embodiment 21. The use of any of the preceding use embodiments, wherein the polypeptide comprises the at least two CBMs are the same or different and are each independently selected.
Embodiment 22. The use of any of the preceding use embodiments, wherein the polypeptide is a heteromultimer.
Embodiment 23. The use of any of the preceding use embodiments, wherein each CBM is independently selected among CBM family 1, 4, 17, 28, 30, 44, 72 and 79, and mixtures thereof; preferably wherein each CBM is a CBM family 1 CBM.
Embodiment 24. The use of any of the preceding use embodiments, wherein the polypeptide comprises three, four, or five CBMs, each from CBM Family 1; preferably comprising three different CBMs, each from CBM Family 1.
Embodiment 25. The use of any of the preceding use embodiments, wherein the CBMs are joined by a linker region.
Embodiment 26. The use of any of the preceding use embodiments, wherein the polypeptide comprises a linker region, which is heterologous to each of the CBMs.
Embodiment 27. The use of any of the preceding use embodiments, wherein each CBM is independently selected among polypeptides having at least 60% sequence identity to one of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 23 e.g. at least 70%, sequence identity, e.g. at least 80% sequence identity, e.g. at least 90% sequence identity; e.g. at least 95%, sequence identity, e.g. at least 96% sequence identity, e.g. at least 97% sequence identity; e.g. at least 98% sequence identity or at least 99% sequence identity, or even 100% sequence identity.
Embodiment 28. The use of any of the preceding use embodiments, wherein each CBM is independently selected from a CBM having the amino acid sequence of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22 or having an amino acid sequence that deviates from one of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 23 by 1, 2, 3, 4, 5, 6, 7, 8 or 9 substitutions, insertions or deletions.
Embodiment 29. The use of any of the preceding use embodiments, the polypeptide comprising four CBMs having the amino acid sequence of SEQ ID NO: 2, SEQ ID NO: 4, SEQ ID NO: 6 and SEQ ID NO: 23, or having an amino acid sequence that deviates from one of SEQ ID NO: 2, SEQ ID NO: 4, SEQ ID NO: 6 and SEQ ID NO: 23 by 1, 2, 3, 4, 5, 6, 7, 8 or 9 substitutions, insertions or deletions.
Embodiment 30. The use of any of the preceding use embodiments, wherein the polypeptide has at least at least 60% sequence identity, e.g., 70% sequence identity, e.g. at least 80% sequence identity, e.g. at least 90% sequence identity; e.g. at least 95%, sequence identity, e.g. at least 96% sequence identity, e.g. at least 97% sequence identity; e.g. at least 98% sequence identity or at least 99% sequence identity, or even 100% sequence identity to the polypeptide of SEQ ID NO: 30, SEQ ID NO: 32, or SEQ ID NO: 38.
Embodiment 31. The use of any of the preceding use embodiments, where the wrinkles are reduced with at least 0.15 units, 0.20 units, 0.25, units, 0.30 units, 0.40 units, 0.5 units when the textile is evaluated by the AATCC Smoothness standard Average SA-value according to AATCC, more preferably at least 0.75 units, e.g. at least 1.0 units, e.g. at least 1.25 units, e.g. at least 1.5 units.
Embodiment 32. The use of any of the preceding use embodiments, wherein the anti-crease effect ratio of test panelists preferring fabrics washed with CBM vs test panelists preferring fabrics washed without CBM is at least 60:40, preferably at least 70:30, preferably at least 80:20 or preferably at least 90:10.
Embodiment 33. The use of any of the preceding use embodiments, wherein the improved softness effect ratio of test panelists preferring fabrics washed with CBM vs test panelists preferring fabrics washed without CBM is at least 60:40, preferably at least 70:30, preferably at least 80:20 or preferably at least 90:10.
Embodiment 34. The use of any of the preceding use embodiments, wherein the fabrics or textiles are selected among cotton containing textiles.
Embodiment 35. A polynucleotide encoding the variant polypeptide of any of embodiments 1-14.
Embodiment 36. A nucleic acid construct comprising the polynucleotide of embodiment 35.
Embodiment 37. An expression vector comprising the polynucleotide of embodiment 35.
Embodiment 38. A host cell comprising a nucleic acid construct according to embodiment 36 and/or an expression vector according to embodiment 37.
Embodiment 39. The host cell of embodiment 38, which is a fungal host cell.
Embodiment 40. The fungal host cell according to embodiment 39, said fungal host cell being a yeast host cell; preferably the yeast host cell is selected from the group consisting of Candida, Hansenula, Kluyveromyces, Pichia, Saccharomyces, Schizosaccharomyces, and Yarrowia cell; more preferably the yeast host cell is selected from the group consisting of Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, and Yarrowia lipolytica cell.
Embodiment 41. The fungal host cell according to embodiment 39, said fungal host cell being a filamentous fungal host cell; preferably the filamentous fungal host cell is selected from the group consisting of Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma cell; more preferably the filamentous fungal host cell is selected from the group consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride cell; even more preferably the filamentous host cell is selected from the group consisting of Aspergillus niger, Aspergillus oryzae, Fusarium venenatum, and Trichoderma reesei; most preferably the filamentous fungal host cell is an Aspergillus niger or an Aspergillus oryzae cell.
Embodiment 42. A method of producing a fusion polypeptide, said method comprising:

a) providing a host cell or a fungal host cell according to any of embodiments 38-41;

b) cultivating said host cell under conditions conducive for expression of the variant polypeptide; and optionally

c) recovering the variant polypeptide.

Embodiment 43. A detergent composition comprising the fusion polypeptide of any of embodiments 1-14.
Embodiment 44. The detergent composition of embodiment 43, further comprising one or more enzymes selected from the group consisting of protease, lipase, cutinase, amylase, carbohydrase, cellulase, pectinase, mannanase, arabinose, galactanase, xylanase, oxidase, nuclease, e.g., laccase, and/or peroxidase.
Embodiment 45. The detergent composition of any of embodiments 43-44, further comprising one or more cleaning composition components such as surfactants, builders, co-builders, polymers, bleaching agents, fabric huing agents and/or perfumes.
Embodiment 46. A laundry booster composition, for use in conjunction with a detergent composition, comprising the polypeptide of any of embodiments 1-14.

### EXAMPLES

### Materials and Methods

Evaluation of wrinkles: AATCC (American Association of Textile Chemists and Colorists) test method 124-TM 124 Smoothness Appearance of Fabrics after Home Laundering (available at members.aatcc.org/store/tm124/533/) (AATCC test method TM 124-2018).
Evaluation of static: AATCC test method 115-Electrostatic Clinging of Fabrics: Fabric-to-Metal Test (available at members.aatcc.org/store/tm115/525/).

### Evaluation by Panellist Preference:

Panelists are asked to select T-shirt part being the less creased. After evaluation, distribution is calculated.

The softness and anti-crease is indicated with X:Y values, wherein X specifies the % of the panelists preferring real items washed with CBM, and Y specifies the % that prefers real item washed without CBM. The sum of the X and Y values is 100%.

### Detergent Compositions

The below mentioned detergent composition can be used in combination with the carbohydrate binding modules described herein for preventing or reducing creases and wrinkles in laundry.

### Composition of Model Detergent B (Liquid):

| Ingredient | Amount (wt %) |
|---|---|
| NaOH, pellets (>99%) | 1.05 |
| Linear alkylbenzenesulfonic acid (LAS) (97%) | 7.20 |
| Sodium laureth sulfate (SLES) (28%) | 10.58 |
| Soy fatty acid (>90%) | 2.75 |
| Coco fatty acid (>99%) | 2.75 |
| AEO; alcohol ethoxylate with 8 mol EO; Lutensol TO 8 (~100%) | 6.60 |
| Triethanol amine (100%) | 3.33 |
| Na-citrate, dihydrate (100%) | 2.00 |
| DTMPA; diethylenetriaminepentakis(methylene)pentakis(phosphonic | 0.48 |
| acid), heptasodium salt (Dequest 2066 C) (~42% as Na7 salt) | |
| MPG (>98%) | 6.00 |
| EtOh, propan-2-ol (90/10%) | 3.00 |
| Glycerol (>99.5) | 1.71 |
| Sodium formate (>95%) | 1.00 |
| PCA (40% as sodium salt) | 0.46 |
| Water up to | 100 |

Final adjustments to the specified pH (pH 8 in the case of Model Detergent B) were done with NaOH or citric acid. Water hardness was adjusted to 15° dH by addition of CaCl₂ and MgCl₂ (Ca²⁺:Mg²⁺=4:1) to the test system.

Composition of Ariel Sensitive White & Color, liquid detergent composition: Aqua, Alcohol Ethoxy Sulfate, Alcohol Ethoxylate, Amino Oxide, Citrid Acid, C12-18 topped palm kernel fatty acid, Protease, Glycosidase, Amylase, Ethanol, 1,2 Propanediol, Sodium Formate, Calcium Chloride, Sodium hydroxide, Silicone Emulsion, Trans-sulphated EHDQ (the ingredients are listed in descending order).
Composition of WFK IEC-A model detergent (powder): Ingredients: Linear sodium alkyl benzene sulfonate 8.8%, Ethoxylated fatty alcohol C12-18 (7 EO) 4.7%, Sodium soap 3.2%, Anti foam DC2-4248S 3.9%, Sodium aluminium silicate zeolite 4A 28.3%, Sodium carbonate 11.6%, Sodium salt of a copolymer from acrylic and maleic acid (Sokalan CP5) 2.4%, Sodium silicate 3.0%, Carboxymethylcellulose 1.2%, Dequest 2066 2.8%, Optical whitener 0.2%, Sodium sulfate 6.5%, Protease 0.4%.
Composition of model detergent A (liquid): Ingredients: 12% LAS, 11% AEO Biosoft N25-7 (NI), 7% AEOS (SLES), 6% MPG (monopropylene glycol), 3% ethanol, 3% TEA, 2.75% cocoa soap, 2.75% soya soap, 2% glycerol, 2% sodium hydroxide, 2% sodium citrate, 1% sodium formiate, 0.2% DTMPA and 0.2% PCA (all percentages are w/w)
Composition of Ariel Actilift (liquid): Ingredients: 5-15% Anionic surfactants; <5% Non-ionic surfactants, Phosphonates, Soap; Enzymes, Optical brighteners, Benzisothiazolinone, Methylisothiazolinone, Perfumes, Alpha-isomethyl ionone, Citronellol, Geraniol, Linalool.
Composition of Ariel Actilift Colour & Style (Ariel Colour & Style): Aqua, Sodium Dodecylbenzenesulfonate, C14-C15 Pareth-7, Sodium Citrate, Propylene Glycol, Sodium Palm Kernelate, Sodium Laureth Sulfate, MEA Dodecylbenzenesulfonage, Sulfated Ethoxylated Hexamethylenediamine Quaternized, Sodium Cumenesulfonate, Perfume, Co-polymer of PEG/Vinyl Acetate, Sodium formate, Hydrogenated Castor Oil, Sodium Diethylenetriamine Pentamethylene Phosphonate, PEG/PPG-10/2 Propylheptyl Ether, Butyophenyl Methylpropional, Polyvinylpyridine-N-Oxide, Sorbitol, Glycerin, Ethanolamine, Sodium Hydroxide, Alpha-Isomethyl lonone, Protease, Calcium Chloride, Geraniol, Linalool, Citronelllol, Tripropylene Glycol, Glycosidase, Benzisothiazolinone, Dimethicone, Glycosidase, Sodium Acetate, Cellulase, Colorant, Glyceryl Stearate, Hydroxyethylcellulose, Silica.
Composition of Ariel Actilift Colour & Style, new pack: Ingredients: Aqua, Sodium Laureth Sulfate, Propylene Glycol, C14-C15 Pareth-7, Sodium citrate, Sodium Palm Kernelate, Alcohol, Sodium Formate, Sulfated Ethoxylated Hexamethylenediamine Quaternized, Sodium Hydroxide, Perfume, Polyvinylpyridine-N-Oxide, Sorbitol, Calcium Chloride, protease, Glycerin, Glucosidase, Glycosidase, Sodium Acetate, Colorant, Cellulase.
Composition of Ariel Actilift Whites & Colours Coolclean, new pack: Ingredients: Aqua, Sodium Laureth Sulfate, Propylene Glycol, C14-C15 Pareth-7, Sodium citrate, Sodium Palm Kernelate, Alcohol, Sodium Formate, Sulfated Ethoxylated Hexamethylenediamine Quaternized, Sodium Hydroxide, Perfume, Sorbitol, Calcium Chloride, protease, Glycerin, Glucosidase, Glycosidase, Sodium Acetate, Colorant, Cellulase.
Composition of Ariel Sensitive White & Color: Ingredients: Aqua, Sodium Laureth Sulfate, Propylene Glycol, C14-C15 Pareth-7, Sodium citrate, Sodium Palm Kernelate, Alcohol, Sodium Formate, Sulfated Ethoxylated Hexamethylenediamine Quaternized, Sodium Hydroxide, Sorbitol, Calcium Chloride, protease, Glycerin, Glycosidase, Sodium Acetate, Cellulase, Silica.
Composition of Ariel Actilift, regular: Aqua, Sodium Dodecylbenzenesulfonate, C14-C15 Pareth-7, Sodium Citrate, Propylene Glycol, Sodium Palm Kernelate, Sodium Laureth Sulfate, MEA Dodecylbenzenesulfonage, Sulfated Ethoxylated Hexamethylenediamine Quaternized, Sodium Cumenesulfonate, Perfume, Co-polymer of PEG/Vinyl Acetate, Sodium formate, C12-C14 Pareth-7, Hydrogenated Castor Oil, Sodium Diethylenetriamine Pentamethylene Phosphonate, PEG/PPG-10/2 Propylheptyl Ether, Butyophenyl Methylpropional, Fluorescent Brightener 9, Sorbitol, Glycerin, Ethanolamine, Sodium Hydroxide, Alpha-Isomethyl lonone, Protease, Calcium Chloride, Geraniol, Linalool, Citronelllol, Tripropylene Glycol, Sodium Chloride, Glycosidase, Benzisothiazolinone, Dimethicone, Glycosidase, Sodium Acetate, Cellulase, Colorant, Glyceryl Stearate, Hydroxyethylcellulose, Silica.
Composition of Persil Small & Mighty (liquid): Ingredients: 15-30% Anionic surfactants, Non-ionic surfacts, 5-15% Soap, <5% Polycarboxylates, Perfume, Phosphates, Optical Brighteners
Composition of Fairy Non Bio (liquid): Ingredients: 15-30% Anionic Surfactants, 5-15% Non-Ionic Surfactants, Soap, Benzisothiazolinone, Methylisothiazolinone, Perfumes
Composition of Model detergent T (powder): Ingredients: 11% LAS, 2% AS/AEOS, 2% soap, 3% AEO, 15.15% sodium carbonate, 3% sodium silicate, 18.75% zeolite, 0.15% chelant, 2% sodium citrate, 1.65% AA/MA copolymer, 2.5% CMC and 0.5% SRP (all percentages are w/w).
Composition of Model detergent X (powder): Ingredients: 16.5% LAS, 15% zeolite, 12% sodium disilicate, 20% sodium carbonate, 1% sokalan, 35.5% sodium sulfate (all percentages are w/w).
Composition of Ariel Actilift (powder): Ingredients: 15-30% Anionic surfactants, <5% Nonionic surfactants, Phosphonates, Polycarboxylates, Zeolites; Enzymes, Perfumes, Hexyl cinnamal.
Composition of Persil Megaperls (powder): Ingredients: 15-30% of the following: anionic surfactants, oxygen-based bleaching agent and zeolites, less than 5% of the following: non-ionic surfactants, phosphonates, polycarboxylates, soap, Further ingredients: Perfumes, Hexyl cinnamal, Benzyl salicylate, Linalool, optical brighteners, Enzymes and Citronellol.
Gain Liquid, Original: Ingredients: Water, Alcohol Ethoxysulfate, Diethylene Glycol, Alcohol Ethoxylate, Ethanolamine, Linear Alkyl Benzene Sulfonate, Sodium Fatty Acids, Polyethyleneimine Ethoxylate, Citric Acid, Borax, Sodium Cumene Sulfonate, Propylene Glycol, DTPA, Disodium Diaminostilbene Disulfonate, Dipropylethyl Tetramine, Sodium Hydroxide, Sodium Formate, Calcium Formate, Dimethicone, Amylase, Protease, Liquitint™, Hydrogenated Castor Oil, Fragrance
Tide Liquid, Original: Ingredients: Linear alkylbenzene sulfonate, propylene glycol, citric acid, sodium hydroxide, borax, ethanolamine, ethanol, alcohol sulfate, polyethyleneimine ethoxylate, sodium fatty acids, diquaternium ethoxysulfate, protease, diethylene glycol, laureth-9, alkyldimethylamine oxide, fragrance, amylase, disodium diaminostilbene disulfonate, DTPA, sodium formate, calcium formate, polyethylene glycol 4000, mannanase, Liquitint™ Blue, dimethicone.
Liquid Tide, Free and Gentle: Water, sodium alcoholethoxy sulfate, propylene glycol, borax, ethanol, linear alkylbenzene sulfonate sodium, salt, polyethyleneimine ethoxylate, diethylene glycol, trans sulfated & ethoxylated hexamethylene diamine, alcohol ethoxylate, linear alkylbenzene sulfonate, MEA salt, sodium formate, sodium alkyl sulfate, DTPA, amine oxide, calcium formate, disodium diaminostilbene, disulfonate, amylase, protease, dimethicone, benzisothiazolinone
Tide Coldwater Liquid, Fresh Scent: Water, alcoholethoxy sulfate, linear alkylbenzene sulfonate, diethylene glycol, propylene glycol, ethanolamine, citric acid, Borax, alcohol sulfate, sodium hydroxide, polyethyleneimine, ethoxylate, sodium fatty acids, ethanol, protease, Laureth-9, diquaternium ethoxysulfate, lauramine oxide, sodium cumene, sulfonate, fragrance, DTPA, amylase, disodium, diaminostilbene, disulfonate, sodium formate, disodium distyrylbiphenyl disulfonate, calcium formate, polyethylene glycol 4000, mannanase, pectinase, Liquitint™ Blue, dimethicone
Tide TOTALCARE™ Liquid, Cool Cotton: Water, alcoholethoxy sulfate, propylene glycol, sodium fatty acids, laurtrimonium chloride, ethanol, sodium hydroxide, sodium cumene sulfonate, citric acid, ethanolamine, diethylene glycol, silicone polyether, borax, fragrance, polyethyleneimine ethoxylate, protease, Laureth-9, DTPA, polyacrylamide quaternium chloride, disodium diaminostilbene disulfonate, sodium formate, Liquitint™ Orange, dipropylethyl tetraamine, dimethicone, cellulase,
Liquid Tide Plus Bleach Alternative™, Vivid White and Bright, Original and Clean Breeze: Water, sodium alcoholethoxy sulfate, sodium alkyl sulfate, MEA citrate, linear alkylbenzene sulfonate, MEA salt, propylene glycol, diethylene glycol, polyethyleneimine ethoxylate, ethanol, sodium fatty acids, ethanolamine, lauramine oxide, borax, Laureth-9, DTPA, sodium cumene sulfonate, sodium formate, calcium formate, linear alkylbenzene sulfonate, sodium salt, alcohol sulfate, sodium hydroxide, diquaternium ethoxysulfate, fragrance, amylase, protease, mannanase, pectinase, disodium diaminostilbene disulfonate, benzisothiazolinone, Liquitint™ Blue, dimethicone, dipropylethyl tetraamine.
Liquid Tide HE, Original Scent: Water, Sodium alcoholethoxy sulfate, MEA citrate, Sodium Alkyl Sulfate, alcohol ethoxylate, linear alkylbenzene sulfonate, MEA salt, sodium fatty acids, polyethyleneimine ethoxylate, diethylene glycol, propylene glycol, diquaternium ethoxysulfate, borax, polyethyleneimine, ethoxylate propoxylate, ethanol, sodium cumene sulfonate, fragrance, DTPA, disodium diaminostilbene disulfonate, Mannanase, cellulase, amylase, sodium formate, calcium formate, Lauramine oxide, Liquitint™ Blue, Dimethicone/polydimethyl silicone.
Tide TOTALCARE HE Liquid, renewing Rain: Water, alcoholethoxy sulfate, linear alkylbenzene sulfonate, alcohol ethoxylate, citric acid, Ethanolamine, sodium fatty acids, diethylene glycol, propylene glycol, sodium hydroxide, borax, polyethyleneimine ethoxylate, silicone polyether, ethanol, protease, sodium cumene sulfonate, diquaternium ethoxysulfate, Laureth-9, fragrance, amylase, DTPA, disodium diaminostilbene disulfonate, disodium distyrylbiphenyl disulfonate, sodium formate, calcium formate, mannanase, Liquitint™ Orange, dimethicone, polyacrylamide quaternium chloride, cellulase, dipropylethyl tetraamine.
Tide liquid HE Free: Water, alcoholethoxy sulfate, diethylene glycol, monoethanolamine citrate, sodium formate, propylene glycol, linear alkylbenzene sulfonates, ethanolamine, ethanol, polyethyleneimine ethoxylate, amylase, benzisothiazolin, borax, calcium formate, citric acid, diethylenetriamine pentaacetate sodium, dimethicone, diquaternium ethoxysulfate, disodium diaminostilbene disulfonate, Laureth-9, mannanase, protease, sodium cumene sulfonate, sodium fatty acids.
Tide Coldwater HE Liquid, Fresh Scent: Water, alcoholethoxy sulfate, MEA Citrate, alcohol sulfate, Alcohol ethoxylate, Linear alkylbenzene sulfonate MEA, sodium fatty acids, polyethyleneimine ethoxylate, diethylene glycol, propylene glycol, diquaternium ethoxysulfate, borax, polyethyleneimine ethoxylate propoxylate, ethanol, sodium cumene sulfonate, fragrance, DTPA, disodium diaminostilbene disulfonate, protease, mannanase, cellulase, amylase, sodium formate, calcium formate, lauramine oxide, Liquitint™ Blue, dimethicone.
Tide for Coldwater HE Free Liquid: Water, sodium alcoholethoxy sulfate, MEA Citrate, Linear alkylbenzene sulfonate: sodium salt, Alcohol ethoxylate, Linear alkylbenzene sulfonate: MEA salt, sodium fatty acids, polyethyleneimine ethoxylate, diethylene glycol, propylene glycol, diquaternium ethoxysulfate, Borax, protease, polyethyleneimine ethoxylate propoxylate, ethanol, sodium cumene sulfonate, Amylase, citric acid, DTPA, disodium diaminostilbene disulfonate, sodium formate, calcium formate, dimethicone.
Tide Simply Clean & Fresh: Water, alcohol ethoxylate sulfate, linear alkylbenzene sulfonate Sodium/Mea salts, propylene glycol, diethylene glycol, sodium formate, ethanol, borax, sodium fatty acids, fragrance, lauramine oxide, DTPA, Polyethylene amine ethoxylate, calcium formate, disodium diaminostilbene disulfonate, dimethicone, tetramine, Liquitint™ Blue.
Tide Pods, Ocean Mist, Mystic Forest, Spring Meadow: Linear alkylbenzene sulfonates, C12-16 Pareth-9, propylene glycol, alcoholethoxy sulfate, water, polyethyleneimine ethoxylate, glycerine, fatty acid salts, PEG-136 polyvinyl acetate, ethylene Diamine disuccinic salt, monoethanolamine citrate, sodium bisulfite, diethylenetriamine pentaacetate sodium, disodium distyrylbiphenyl disulfonate, calcium formate, mannanase, exyloglucanase, sodium formate, hydrogenated castor oil, natalase, dyes, termamyl, subtilisin, benzisothiazolin, perfume.
Tide to Go: Deionized water, Dipropylene Glycol Butyl Ether, Sodium Alkyl Sulfate, Hydrogen Peroxide, Ethanol, Magnesium Sulfate, Alkyl Dimethyl Amine Oxide, Citric Acid, Sodium Hydroxide, Trimethoxy Benzoic Acid, Fragrance.
Tide Stain Release Liquid: Water, Alkyl Ethoxylate, Linear Alkylbenzenesulfonate, Hydrogen Peroxide, Diquaternium Ethoxysulfate, Ethanolamine, Disodium Distyrylbiphenyl Disulfonate, tetrabutyl Ethylidinebisphenol, F&DC Yellow 3, Fragrance.
Tide Stain Release Powder: Sodium percarbonate, sodium sulfate, sodium carbonate, sodium aluminosilicate, nonanoyloxy benzene sulfonate, sodium polyacrylate, water, sodium alkylbenzenesulfonate, DTPA, polyethylene glycol, sodium palmitate, amylase, protease, modified starch, FD&C Blue 1, fragrance.
Tide Stain Release, Pre Treater Spray: Water, Alkyl Ethoxylate, MEA Borate, Linear Alkylbenzenesulfonate, Propylene Glycol, Diquaternium Ethoxysulfate, Calcium Chlorideenzyme, Protease, Ethanolamine, Benzoisothiazolinone, Amylase, Sodium Citrate, Sodium Hydroxide, Fragrance.
Tide to Go Stain Eraser: Water, Alkyl Amine Oxide, Dipropylene Glycol Phenyl Ether, Hydrogen Peroxide, Citric Acid, Ethylene Diamine Disuccinic Acid Sodium salt, Sodium Alkyl Sulfate, Fragrance.
Tide boost with Oxi: Sodium bicarbonate, sodium carbonate, sodium percarbonate, alcohol ethoxylate, sodium chloride, maleic/acrylic copolymer, nonanoyloxy benzene sulfonate, sodium sulfate, colorant, diethylenetriamine pentaacetate sodium salt, hydrated aluminosilicate (zeolite), polyethylene glycol, sodium alkylbenzene sulfonate, sodium palmitate, starch, water, fragrance.
Tide Stain Release boost Duo Pac: Polyvinyl Alcoholpouch film, wherein there is packed a liquid part and a powder part: Liquid Ingredients: Dipropylene Glycol, diquaternium Ethoxysulfate, Water, Glycerin, Liquitint™ Orange, Powder Ingredients: sodium percarbonate, nonanoyloxy benzene sulfonate, sodium carbonate, sodium sulfate, sodium aluminosilicate, sodium polyacrylate, sodium alkylbenzenesulfonate, maleic/acrylic copolymer, water, amylase, polyethylene glycol, sodium palmitate, modified starch, protease, glycerine, DTPA, fragrance.
Tide Ultra Stain Release: Water, sodium alcoholethoxy sulfate, linear alkyl benzene sulfonate, sodium/MEA salts, MEA citrate, propylene glycol, polyethyleneimine ethoxylate, ethanol, diethylene glycol, polyethyleneimine propoxyethoxylate, sodium fatty acids, protease, borax, sodium cumene sulfonate, DTPA, fragrance, amylase, disodium diaminostilbene disulfonate, calcium formate, sodium formate, gluconase, dimethicone, Liquitint™ Blue, mannanase.
Ultra Tide with a Touch of Downy® Powdered Detergent, April Fresh/Clean Breeze/April Essence: Sodium Carbonate, Sodium Aluminosilicate, Sodium Sulfate, Linear Alkylbenzene Sulfonate, Bentonite, Water, Sodium Percarbonate, Sodium Polyacrylate, Silicate, Alkyl Sulfate, Nonanoyloxybenzenesulfonate, DTPA, Polyethylene Glycol 4000, Silicone, Ethoxylate, fragrance, Polyethylene Oxide, Palmitic Acid, Disodium Diaminostilbene Disulfonate, Protease, Liquitint™ Red, FD&C Blue 1, Cellulase.
Ultra Tide with a Touch of Downy Clean Breeze: Water, sodium alcoholethoxy sulfate, MEA citrate, linear alkyl benzene sulfonate: sodium/MEA salts, propylene glycol, polyethyleneimine ethoxylate, ethanol, diethylene glycol, polyethyleneimine, propoxyethoxylate, diquaternium ethoxysulfate, alcohol sulfate, dimethicone, fragrance, borax, sodium fatty acids, DTPA, protease, sodium bisulfite, disodium diaminostilbene disulfonate, amylase, gluconase, castor oil, calcium formate, MEA, styrene acrylate copolymer, sodium formate, Liquitint™ Blue.
Ultra Tide with Downy Sun Blossom: Water, sodium alcoholethoxy sulfate, MEA citrate, linear alkyl benzene sulfonate: sodium/MEA salts, propylene glycol, ethanol, diethylene glycol, polyethyleneimine propoxyethoxylate, polyethyleneimine ethoxylate, alcohol sulfate, dimethicone, fragrance, borax, sodium fatty acids, DTPA, protease, sodium bisulfite, disodium diaminostilbene disulfonate, amylase, castor oil, calcium formate, MEA, styrene acrylate copolymer, propanaminium propanamide, gluconase, sodium formate, Liquitint™ Blue.
Ultra Tide with Downy April Fresh/Sweet Dreams: Water, sodium alcoholethoxy sulfate, MEA citrate, linear alkyl benzene sulfonate: sodium/MEA salts, propylene glycol, polyethyleneimine ethoxylate, ethanol, diethylene glycol, polyethyleneimin propoxyethoxylate, diquaternium ethoxysulfate, alcohol sulfate, dimethicone, fragrance, borax, sodium fatty acids, DTPA, protease, sodium bisulfite, disodium diaminostilbene disulfonate, amylase, gluconase, castor oil, calcium formate, MEA, styrene acrylate copolymer, propanaminium propanamide, sodium formate, Liquitint™ Blue.
Ultra Tide Free Powdered Detergent: Sodium Carbonate, Sodium Aluminosilicate, Alkyl Sulfate, Sodium Sulfate, Linear Alkylbenzene Sulfonate, Water, Sodium polyacrylate, Silicate, Ethoxylate, Sodium percarbonate, Polyethylene Glycol 4000, Protease, Disodium Diaminostilbene Disulfonate, Silicone, Cellulase.
Ultra Tide Powdered Detergent, Clean Breeze/Spring Lavender/mountain Spring: Sodium Carbonate, Sodium Aluminosilicate, Sodium Sulfate, Linear Alkylbenzene Sulfonate, Alkyl Sulfate, Sodium Percarbonate, Water, Sodium Polyacrylate, Silicate, Nonanoyloxybenzenesulfonate, Ethoxylate, Polyethylene Glycol 4000, Fragrance, DTPA, Disodium Diaminostilbene Disulfonate, Palmitic Acid, Protease, Silicone, Cellulase.
Ultra Tide HE (high Efficiency) Powdered Detergent, Clean Breeze: Sodium Carbonate, Sodium Aluminosilicate, Sodium Sulfate, Linear Alkylbenzene Sulfonate, Water, Nonanoyloxybenzenesulfonate, Alkyl Sulfate, Sodium Polyacrylate, Silicate, Sodium Percarbonate, Ethoxylate, Polyethylene Glycol 4000, Fragrance, DTPA, Palmitic Acid, Disodium Diaminostilbene Disulfonate, Protease, Silicone, Cellulase.
Ultra Tide Coldwater Powdered Detergent, Fresh Scent: Sodium Carbonate, Sodium Aluminosilicate, Sodium Sulfate, Sodium Percarbonate, Alkyl Sulfate, Linear Alkylbenzene Sulfonate, Water, Nonanoyloxybenzenesulfonate, Sodium Polyacrylate, Silicate, Ethoxylate, Polyethylene Glycol 4000, DTPA, Fragrance, Natalase, Palmitic Acid, Protease, Disodium, Diaminostilbene Disulfonate, FD&C Blue 1, Silicone, Cellulase, Alkyl Ether Sulfate.
Ultra Tide with bleach Powdered Detergent, Clean Breeze: Sodium Carbonate, Sodium Aluminosilicate, Sodium Sulfate, Linear Alkylbenzene Sulfonate, Sodium Percarbonate, Nonanoyloxybenzenesulfonate, Alkyl Sulfate, Water, Silicate, Sodium Polyacrylate, Ethoxylate, Polyethylene Glycol 4000, Fragrance, DTPA, Palmitic Acid, Protease, Disodium Diaminostilbene Disulfonate, Silicone, FD&C Blue 1, Cellulase, Alkyl Ether Sulfate.
Ultra Tide with Febreeze Freshness™ Powdered Detergent, Spring Renewal: Sodium Carbonate, Sodium Aluminosilicate, Sodium Sulfate, Linear Alkylbenzene Sulfonate, Sodium Percarbonate, Alkyl Sulfate, Water, Sodium Polyacrylate, Silicate, Nonanoyloxybenzenesulfonate, Ethoxylate, Polyethylene Glycol 4000, DTPA, Fragrance, Cellulase, Protease, Disodium Diaminostilbene Disulfonate, Silicone, FD&C Blue 1.
Liquid Tide Plus with Febreeze Freshness—Sport HE Active Fresh: Water, Sodium alcoholethoxy sulfate, MEA citrate, linear alkylbenzene sulfonate, sodium salt, linear alkylbenzene sulfonate: MEA salt, alcohol ethoxylate, sodium fatty acids, propylene glycol, diethylene glycol, polyethyleneimine ethoxylate propoxylate, diquaternium ethoxysulfate, Ethanol, sodium cumene sulfonate, borax, fragrance, DTPA, Sodium bisulfate, disodium diaminostilbene disulfonate, Mannanase, cellulase, amylase, sodium formate, calcium formate, Lauramine oxide, Liquitint™ Blue, Dimethicone/polydimethyl silicone.
Tide Plus Febreeze Freshness Spring & Renewal: Water, sodium alcoholethoxy sulfate, linear alkyl benzene sulfonate: sodium/MEA salts, MEA citrate, propylene glycol, polyethyleneimine ethoxylate, fragrance, ethanol, diethylene glycol, polyethyleneimine propoxyethoxylate, protease, alcohol sulfate, borax, sodium fatty acids, DTPA, disodium diaminostilbene disulfonate, MEA, mannanase, gluconase, sodium formate, dimethicone, Liquitint™ Blue, tetramine.
Liquid Tide Plus with Febreeze Freshness, Sport HE Victory Fresh: Water, Sodium alcoholethoxy sulfate, MEA citrate, linear alkylbenzene sulfonate, sodium salt, linear alkylbenzene sulfonate: MEA salt, alcohol ethoxylate, sodium fatty acids, propylene glycol, diethylene glycol, polyethyleneimine ethoxylate propoxylate, diquaternium ethoxysulfate, ethanol, sodium cumene sulfonate, borax, fragrance, DTPA, Sodium bisulfate, disodium diaminostilbene disulfonate, Mannanase, cellulase, amylase, sodium formate, calcium formate, Lauramine oxide, Liquitint™ Blue, Dimethicone/polydimethyl silicone.

### Tide Vivid White+Bright Powder, Original: Sodium Carbonate, Sodium Aluminosilicate, Sodium Sulfate, Linear Alkylbenzene Sulfonate, Sodium Percarbonate, Nonanoyloxybenzenesulfonate, Alkyl Sulfate, Water, Silicate, Sodium Polyacrylate

### Ethoxylate, Polyethylene Glycol 4000, Fragrance, DTPA, Palmitic Acid, Protease, Disodium Diaminostilbene Disulfonate, Silicone, FD&C Blue 1, Cellulase, Alkyl Ether Sulfate.

Hey Sport Tex Wash Detergent: Aqua, dodecylbenzenesulfonssure, laureth-11, peg-75 lanolin, propylene glycol, alcohol denat., potassium soyate, potassium hydroxide, disodium cocoamphodiacetate, ethylendiamine triacetate cocosalkyl acetamide, parfum, zinc ricinoleate, sodium chloride, benzisothiazolinone, methylisothiazolinone, ci 16255, benzyl alcohol.

The products named Tide, Ariel, Gain and Fairy are commercially available products supplied by Procter & Gamble. The products named Persil are commercially available products supplied by Unilever and Henkel. The products named Hey Sport are commercially available products supplied by Hey Sport.

TABLE 1

| Ingredient | Amount (in wt %) |
|---|---|
| Anionic detersive surfactant (such as alkyl benzene sulphonate, alkyl | from 8% to 15% |
| ethoxylated sulphate and mixtures | |
| Non-ionic detersive surfactant (such as alkyl ethoxylated alcohol) | from 0.5% to 4% |
| Cationic detersive surfactant (such as quaternary ammonium | from 0 to 4% |
| compounds) | |
| Other detersive surfactant (such as zwiterionic detersive surfactants, | from 0% to 4% |
| amphoteric surfactants and mixtures thereof) | |
| Carboxylate polymer (such as co-polymers of maleic acid and acrylic | from 1% to 4% |
| acid) | |
| Polyethylene glycol polymer (such as a polyethylene glycol polymer | from 0.5% to 4% |
| comprising poly vinyl acetate side chains) | |
| Polyester soil release polymer (such as Repel-o-tex from and/or Texcare | from 0.1 to 2% |
| polymers) | |
| Cellulosic polymer (such as carboxymethyl cellulose, methyl cellulose | from 0.5% to 2% |
| and combinations thereof) | |
| Other polymer (such as amine polymers, dye transfer inhibitor polymers, | from 0% to 4% |
| hexamethylenediamine derivative polymers, and mixtures thereof) | |
| Zeolite builder and phosphate builder (such as zeolite 4A and/or sodium | from 0% to 4 wt % |
| tripolyphosphate) | |
| Other builder (such as sodium citrate and/or citric acid) | from 0% to 3% |
| Carbonate salt (such as sodium carbonate and/or sodium bicarbonate) | from 15% to 30% |
| Silicate salt (such as sodium silicate) | from 0% to 10% |
| Filler (such as sodium sulphate and/or bio-fillers) | from 10% to 40% |
| Source of available oxygen (such as sodium percarbonate) | from 10% to 20% |
| Bleach activator (such as tetraacetylethylene diamine (TAED) and/or | from 2% to 8% |
| nonanoyloxybenzenesulphonate (NOBS) | |
| Bleach catalyst (such as oxaziridinium-based bleach catalyst and/or | from 0% to 0.1% |
| transition metal bleach catalyst) | |
| Other bleach (such as reducing bleach and/or pre- formed peracid) | from 0% to 10% |
| Chelant (such as ethylenediamine-N′N′-disuccinic acid (EDDS) and/or | from 0.2% to 1% |
| hydroxyethane diphosphonic acid(HEDP) | |
| Photobleach (such as zinc and/or aluminium sulphonated | from 0% to 0.1% |
| phthalocyanine) | |
| Hueing agent (such as direct violet 99, acid red 52, acid blue 80, direct | from 0% to 1% |
| violet 9, solvent violet 13 and any combination thereof) | |
| Brightener (such as brightener 15 and/or brightener 49) | from 0.1% to 0.4% |
| Protease such as those mentioned under the heading “proteases” e.g. | from 0.1% to 0.4% |
| Savinase, Savinase Ultra, Ovozyme, Kannase, Liquanase, Polarazyme, | |
| Purafect, Properase, FN3, FN4 and any combination thereof) | |
| Amylase (such as Termamyl, Termamyl ultra Natalase, Optisize, | from 0.05% to 0.2% |
| Stainzyme, Stainzyme Plus, and any combination thereof) | |
| Cellulase (such as Carezyme and/or Celluclean) | from 0.05% to 0.2% |
| Lipase (such as Lipex, Lipolex, Lipoclean and any combination thereof) | from 0.2 to 1% |
| Other enzyme (such as xyloglucanase, cutinase, pectate lyase, | from 0% to 2% |
| mannanase, bleaching enzyme) | |
| Fabric softener (such as montmorillonite clay and/or polydimethylsiloxane | from 0% to 4% |
| (PDMS) | |
| Flocculant (such as polyethylene oxide) | from 0% to 1% |
| Suds suppressor (such as silicone and/or fatty acid) | from 0% to 0.1% |
| Perfume (such as perfume microcapsule, spray-on perfume, starch | from 0.1% to 1% |
| encapsulated perfume accords, perfume loaded zeolite, and any | |
| combination thereof) | |
| Aesthetics (such as coloured soap rings and/or coloured | from 0% to 1% |
| speckles/noodles) | |
| Miscellaneous | balance |

TABLE 2

| Ingredient | Amount |
|---|---|
| Carboxyl group-containing polymer (comprising from about 60% to about | from about 0.5 wt % |
| 70% by mass of an acrylic acid-based monomer (A); and from about 30% | to about 1.5 wt % |
| to about 40%) by mass of a sulfonic acid group-containing monomer (B); | |
| and wherein the average molecular weight is from about 23,000 to about | |
| 50,000 preferably in the range of from about 25,000 to about | |
| 38,000 as described in WO2014032269. | |
| Amylase (Stainzyme Plus(R), having an enzyme activity of 14 mg active | from about 0.1 wt % |
| enzyme/g) | to about 0.5 wt % |
| Anionic detersive surfactant (such as alkyl benzene sulphonate, alkyl | from about 8 wt % |
| ethoxylated sulphate and mixtures thereof) | to about 15 wt % |
| Non-ionic detersive surfactant (such as alkyl ethoxylated alcohol) | from about 0.5 wt % |
| | to 4 wt % |
| Cationic detersive surfactant (such as quaternary ammonium | from about 0 wt % |
| compounds) | to about 4 wt % |
| Other detersive surfactant (such as zwiterionic detersive surfactants, | from about 0 wt % |
| amphoteric surfactants and mixtures thereof) | to 4 wt % |
| Carboxylate polymer (such as co-polymers of maleic acid and acrylic | from about 1 wt % |
| acid) | to about 4 wt % |
| Polyethylene glycol polymer (such as a polyethylene glycol polymer | from about 0 wt % |
| comprising poly vinyl acetate side chains) | to about 4 wt % |
| Polyester soil release polymer (such as Repel-O- Tex(R) and/or | from about 0.1 wt % |
| Texcare(R) polymers) | to about 2 wt % |
| Cellulosic polymer (such as carboxymethyl cellulose, methyl cellulose | from about 0.5 wt % |
| and combinations thereof) | to about 2 wt % |
| Other polymer (such as amine polymers, dye transfer inhibitor polymers, | from about 0 wt % |
| hexamethylenediamine derivative polymers, and mixtures thereof) | to about 4 wt % |
| Zeolite builder and phosphate builder (such as zeolite 4A and/or sodium | from about 0 wt % |
| tripolyphosphate) | to about 4 wt % |
| Other builder (such as sodium citrate and/or citric acid) | from about 0 wt % |
| | to about 3 wt % |
| Carbonate salt (such as sodium carbonate and/or sodium bicarbonate) | from about 15 t % |
| | to about 30 wt % |
| Silicate salt (such as sodium silicate) | from about 0 wt % |
| | to about 10 wt % |
| Filler (such as sodium sulphate and/or bio-fillers) | from about 10 wt % |
| | to about 40 wt % |
| Source of available oxygen (such as sodium percarbonate) | from about 10 wt % |
| | to about 20 wt % |
| Bleach activator (such as tetraacetylethylene diamine (TAED) and/or | from about 2 wt % |
| nonanoyloxybenzenesulphonate (NOBS) | to about 8 wt % |
| Bleach catalyst (such as oxaziridinium-based bleach catalyst and/or | from about 0 wt % |
| transition metal bleach catalyst) | to about 0.1 wt % |
| Other bleach (such as reducing bleach and/or pre formed peracid) | from about 0 wt % |
| | to about 10 wt % |
| Chelant (such as ethylenediamine-N′N′-disuccinic acid (EDDS) and/or | from about 0.2 wt % |
| hydroxyethane diphosphonic acid (HEDP) | to about 1 wt % |
| Photobleach (such as zinc and/or aluminium sulphonated | from about 0 wt % |
| phthalocyanine) | to about 0.1 wt % |
| Hueing agent (such as direct violet 99, acid red 52, acid blue 80, direct | from about 0 wt % |
| violet 9, solvent violet 13 and any combination thereof) | to about 0.5 wt % |
| Brightener (such as brightener 15 and/or brightener 49) | from about 0.1 wt % |
| | to about 0.4 wt % |
| Protease such as those mentioned under the heading “proteases” e.g. | from about 0.1 wt % |
| Savinase, Savinase Ultra, Ovozyme, Kannase, Liquanase, Polarazyme, | to about 1.5 wt % |
| Purafect, Properase, FN3, FN4 and any combination thereof, typically | |
| having an enzyme activity of from about 20 mg to about 100 mg active | |
| exzyme/g) | |
| Amylase (such as Termamyl(R), Termamyl Ultra(R), Natalase(R), | from about 0.05 wt % |
| Optisize HT Plus(R), Powerase(R), Stainzyme(R) and any combination | to about 0.2 wt % |
| thereof, typically having an enzyme activity of from about 10 mg | |
| to about 50 mg active enzyme/g) | |
| Cellulase (such as Carezyme(R), Celluzyme(R) and/or Celluclean(R), | from about 0.05 wt % |
| typically having an enzyme activity of about from 10 to 50 mg active | to 0.5 wt % |
| enzyme/g) | |
| Lipase (such as Lipex(R), Lipolex(R), Lipoclean(R) and any combination | from about 0.2 wt % |
| thereof, typically having an enzyme activity of from about 10 mg to about | to about 1 wt % |
| 50 mg active enzyme/g) | |
| Other enzyme (such as xyloglucanase (e.g., Whitezyme(R)), cutinase, | from 0 wt % to 2 wt % |
| pectate lyase, mannanase, bleaching enzyme, typically having an | |
| enzyme activity of from about 10 mg to about 50 mg active enzyme/g) | |
| Fabric softener (such as montmorillonite clay and/or polydimethylsiloxane | from 0 wt % to 15 wt % |
| (PDMS)) | |
| Flocculant (such as polyethylene oxide) | from 0 wt % to 1 wt % |
| Suds suppressor (such as silicone and/or fatty acid) | from 0 wt % to 0.1 wt % |
| Perfume (such as perfume microcapsule, spray-on perfume, starch | from 0.1 wt % to 1 wt % |
| encapsulated perfume accords, perfume loaded zeolite, and any | |
| combination thereof) | |
| Aesthetics (such as colored soap rings and/or colored speckles/noodles) | from 0 wt % to 1 wt % |
| Miscellaneous | Balance |

All enzyme levels expressed as rug active enzyme protein per 100 g detergent composition.

Surfactant ingredients can be obtained from BASF, Ludwigshafen, Germany (Lutensol®); Shell Chemicals, London, UK; Stepan, Northfield, Ill., USA; Huntsman, Huntsman, Salt Lake City, Utah, USA; Clariant, Sulzbach, Germany (Praepagen®).

Sodium tripolyphosphate can be obtained from Rhodia, Paris, France.
Zeolite can be obtained from Industrial Zeolite (UK) Ltd, Grays, Essex, UK.
Citric acid and sodium citrate can be obtained from Jungbunzlauer, Basel, Switzerland.
NOBS is sodium nonanoyloxybenzenesulfonate, supplied by Eastman, Batesville, Ark., USA.
TAED is tetraacetylethylenediamine, supplied under the Peractive®brand name by Clariant GmbH, Sulzbach, Germany.
Sodium carbonate and sodium bicarbonate can be obtained from Solvay, Brussels, Belgium.
Polyacrylate, polyacrylate/maleate copolymers can be obtained from BASF, Ludwigshafen, Germany.
Repel-O-Tex®can be obtained from Rhodia, Paris, France.
Texcare®can be obtained from Clariant, Sulzbach, Germany. Sodium percarbonate and sodium carbonate can be obtained from Solvay, Houston, Tex., USA.
Na salt of Ethylenediamine-N,N′-disuccinic acid, (S,S) isomer (EDDS) was supplied by Octel, Ellesmere Port, UK.
Hydroxy ethane di phosphonate (HEDP) was supplied by Dow Chemical, Midland, Mich., USA. Enzymes Savinase®, Savinase®Ultra, Stainzyme®Plus, Lipex®, Lipolex®, Lipoclean®, Celluclean®, Carezyme®, Natalase®, Stainzyme®, Stainzyme®Plus, Termamyl®, Termamyl®ultra, and Mannaway®can be obtained from Novozymes, Bagsvaerd, Denmark.
Enzymes Purafect®, FN3 and FN4 can be obtained from DuPont International
Inc., Palo Alto, Calif., US. Direct violet 9 and 99 can be obtained from BASF DE, Ludwigshafen, Germany. Solvent violet 13 can be obtained from Ningbo Lixing Chemical Co., Ltd. Ningbo, Zhejiang, China. Brighteners can be obtained from Ciba Specialty Chemicals, Basel, Switzerland. All percentages and ratios are calculated by weight unless otherwise indicated. All percentages and ratios are calculated based on the total composition unless otherwise indicated. It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Wash Assays

### Launder-O-Meter (LOM) Model Wash System

The Launder-O-Meter (LOM) is a medium scale model wash system that can be applied to test up to 20 different wash conditions simultaneously. A LOM is basically a large temperature controlled water bath with 20 closed metal beakers rotating inside it. Each beaker constitutes one small washing machine and during an experiment, each will contain a solution of a specific detergent/enzyme system to be tested along with the soiled and unsoiled fabrics it is tested on. Mechanical stress is achieved by the beakers being rotated in the water bath and by including metal balls in the beaker.

The LOM model wash system is mainly used in medium scale testing of detergents and enzymes at European wash conditions. In a LOM experiment, factors such as the ballast to soil ratio and the fabric to wash liquor ratio can be varied. Therefore, the LOM provides the link between small scale experiments, such as AMSA and mini-wash, and the more time consuming full scale experiments in front loader washing machines.

### Mini Launder-O-Meter (MiniLOM) Model Wash System

MiniLOM is a modified mini wash system of the Launder-O-Meter (LOM), which is a medium scale model wash system that can be applied to test up to 20 different wash conditions simultaneously. A LOM or is basically a large temperature controlled water bath with 20 closed metal beakers rotating inside it. Each beaker constitutes one small washing machine and during an experiment, each will contain a solution of a specific detergent/enzyme system to be tested along with the soiled and unsoiled fabrics it is tested on. Mechanical stress is achieved by the beakers being rotated in the water bath and by including metal balls in the beaker.

The LOM model wash system is mainly used in medium scale testing of detergents and enzymes at European wash conditions. In a LOM experiment, factors such as the ballast to soil ratio and the fabric to wash liquor ratio can be varied. Therefore, the LOM provides the link between small scale experiments, such as AMSA and mini-wash, and the more time consuming full scale experiments in front loader washing machines.

In miniLOM, washes are performed in 50 ml test tubes placed in Stuart rotator.

### Terg-O-Tometer (TOM) Wash Assay

The Terg-O-tometer (TOM) is a medium scale model wash system that can be applied to test 12 different wash conditions simultaneously. A TOM is basically a large temperature controlled water bath with up to 12 open metal beakers submerged into it. Each beaker constitutes one small top loader style washing machine and during an experiment, each of them will contain a solution of a specific detergent/enzyme system and the soiled and unsoiled fabrics its performance is tested on. Mechanical stress is achieved by a rotating stirring arm, which stirs the liquid within each beaker. Because the TOM beakers have no lid, it is possible to withdraw samples during a TOM experiment and assay for information on-line during wash.

The TOM model wash system is mainly used in medium scale testing of detergents and enzymes at US or LA/AP wash conditions, as well as for EU conditions. In a TOM experiment, factors such as the ballast to soil ratio and the fabric to wash liquor ratio can be varied. Therefore, the TOM provides the link between small scale experiments and the more time consuming full scale experiments in top loader washing machines.

### Production of Variants

Expression constructs were constructed by preparing a shuttle plasmid comprising the nucleotide sequence encoding the CBM in operation connection with an Aspergillus promoter, signal sequence and Kex cleavage site and terminator, and further comprising an amdS gene for amdS selection in Aspergillus. The promoter used for the CBM production is further described in WO2003/008575. The correctness of the constructs was confirmed by sequencing.
Aspergillus transformation: An Aspergillus (e.g. A. niger or A. oryzae) laboratory strain is transformed with the expression constructs and grown under inductive conditions for expression of the CBM.
Recovery of CBM: After growing the transformed Aspergillus, the CBM is purified from the supernatant using standard chromatographic methods.

### Example 1. Preparation of CBM Monomers

Three CBMs, belonging to the CBM1 family, were prepared as described under Methods and Materials
CBM1-1 was derived from Fusarium longipes GH10 polypeptide and was encoded by the nucleotide sequence:

(SEQ ID NO: 1)
cagtcccccatctggggacagtgtggtggaaacggatggactggtgcaaca
acatgtcagtccggactcaagtgtgagaaagtgaacgattggtactaccag
tgtgtcccctaa

and had the amino acid sequence:

(SEQ ID NO: 2)
QSPIWGQCGGNGWTGATTCQSGLKCEKVNDWYYQCVP

CBM1-2 was derived from Fusarium longipes GH6 polypeptide and was encoded by the nucleotide sequence:

(SEQ ID NO: 3)
gcaccggtcgaagaacgacagtcgtgttcgaacggagtctgggcacagtgt
ggtggtcagaactggtcgggtacaccctgttgtacatccggcaacacatgt
gtcaaaatcaacgacttctactcgcagtgtcagcctggctaa

and had the amino acid sequence:

(SEQ ID NO: 4)
APVEERQSCSNGVWAQCGGQNWSGTPCCTSGNTCVKINDFYSQCQPG

CBM1-3 was derived from Aspergillus clavatus carbohydrate esterase CE1 polypeptide and was encoded by the nucleotide sequence:

(SEQ ID NO: 5)
cagcagtccctctatggccagtgtggaggtaacggctggtccggacccaca
gagtgtacagcaggagcatgttgtcaggtccagaacccgtggtattcccag
tgtctccctggcgattgttaa

and had the amino acid sequence:

(SEQ ID NO: 6)
QQSLYGQCGGNGWSGPTECTAGACCQVQNPWYSQCLPGDC

Additional CBMs of various CBM families were prepared. The overall cloning and transformation methods are the same as in the Materials and Methods section, but the genes encoding for the recombinant CBMs were codon-optimized for Aspergillus oryzae and synthesized by GeneArt. The signal peptide sequence MKLSWLVAAALTAASVVSA (SEQ ID NO: 21) was used for secretion of the recombinant CBMs.
CBM79 was derived from Ruminococcus flavefaciens GH9 endoglucanase polypeptide and was encoded by the nucleotide sequence of SEQ ID NO: 7 and has the amino acid sequence:

(SEQ ID NO: 8)
DGYTIKPNKKVTYSALGEDERMIGFSYKDFGISSSEKITEVQVNI-SANKN
IGKYVGQFGTSTTDSANGYWAMGDEITQSISGNSGTITWKVPSDISSIIQT
QYGGEIKFGVWWIDCDEFTIDSVVLK

CBM72 was derived from unidentified microorganism GH5 endoglucanase polypeptide and was encoded by the nucleotide sequence of SEQ ID NO: 9 and has the amino acid sequence:

(SEQ ID NO: 10)
GYKYPTADDFEIVYDISYNDEWSELFLFGSWDRTAVNLSGYKGIRVEMDKA
YGNKLQIKVYG-DKKSGTDFNEQYAPLSDTSASTTVDFDTSILGSTFWGVT
LQTNSGALTATLKEAKLIKADGTEEPASVTAAWGCTVTAKSTPKPTGIHAI
QLIKTEADGAIYNLQGQRVQNPQKGIYIQNGKKYVMK

CBM44 was derived from Hungateiclostridium thermocellum GH9 endoglucanase polypeptide and was encoded by the nucleotide sequence of SEQ ID NO: 11 and has the amino acid sequence:

(SEQ ID NO: 12)
GTLGGFTTSGTNATGVVVNTTEKAFKGERGLKWTVTSEGEGTAELKLDGGTIVVPGTT-
MTFRIWIPSGAPIAAIQPYIMPHTPDWSEVLWNSTWKGYTMVKTDDWNEITLTLPEDVDPTWP
QQMGIQVQTIDEGEFTIYVDAIDW

The produced protein contains 19.9% of protein with sequence of SEQ ID NO: 12 and 80.1% of protein having the mutation G134S.
CBM30 was derived from Clostridium cellulovorans GH9 endoglucanase polypeptide and was encoded by the nucleotide sequence of SEQ ID NO: 13 and has the amino acid sequence:

(SEQ ID NO: 14)
KLMDLEVFKSASITGWSGSAGGELEVASDSNLPIDTSATYNGLPSLRLNVTKASAQWWS-
SLLTLRGWCTQDLTQYLANGYLEFNVKGKVGGEDFQIGLQDQTHERAAGDSVTSVKSIKNYVN
ISTNWQHVKIPLKDIMGPSTGFDPTTARCINIVKGSSEIFTAWINDLKITSTDNEK

A heterodimer comprising CBM17 and CBM28 was derived from Clostridium cellulovorans GH5 endoglucanase polypeptide and was encoded by the nucleotide sequence of SEQ ID NO: 15 and has the amino acid sequence:

(SEQ ID NO: 16)
LWDFNDGTKQGFGVNGDSPVEDVVIENEAGALKLSGLDASNDVSEGNYWANARLSADG-
WGKSVDILGAEKLTMDVIVDEPTTVSIAAIPQGPSANWVNPNRAIKVEPTNFVPLGDKFKAELTI
TSADSPSLEAIAMHAENNNINNIILFVGTEGADVIYLDNIKVIG-
TEVEIPVVHDPKGEAVLPSVFEDGTRQGWDWAGESGVKTALTIEEANGSNALSWEFGYPEVK
PSDNWATAPRLDFWKSDLVRGENDYVTFDFYLDPVRATEGAMNINLVFQPPTNGYWVQAP-
KTYTINFDELEEANQVNGLYHYEVKINVRDITNIQDDTLLRNMMIIFADVESDFAGRVFVDNVRF
EGAATTE

The produced protein also includes protein having the mutation V174M.
LWDFNDGTKQGFGVNGDSPVEDVVIENEAGALKLSGLDASNDVSEGNYWANARLSADGWGKSVDILGAEKLTMDVIVDEPTTVSIAAIPQGPSANWVNPNRAIKVEPTNFVPLGDKFKAELTI TSADSPSLEAIAMHAENNNINNIILFVGTEGADVIYLDNIKVI (SEQ ID NO: 17) and GTEVEIPVVHDPKGEAVLPSVFEDGTRQGWDWAGESGVKTALTIEEANGSNALSWEFGYPEVKPSDNWATAPRLDFWKSDLVRGENDYVTFDFYLDPVRATEGAMNINLVFQPPT NGYWVQAPKTYTINFDELEEANQVNGLYHYEVKINVRDITNIQDDTLLRNMMIIFADVESDFAGRVFVDNVRFEGAATTE (SEQ ID NO: 18) correspond to the CBM17 and CBM28 portions, respectively.
CBM4 was derived from Cellulomonas fimi GH9 endoglucanase polypeptide and was encoded by the nucleotide sequence of SEQ ID NO: 19 and has the amino acid sequence:

(SEQ ID NO: 20)
ASPIGEGTFDDGPEGWVAYGTDGPLDTSTGALCVAVPAGSAQYGVGVVLNGVAIEEGTTYTL-
RYTATASTDVTVRALVGQNGAPYGTVLDTSPALTSEPRQVTETFTASATYPATPAADDPEGQIA
FQLGGFSADAWTFCLDDVALDSEVELLP

### Example 2. Preparation of CBM1-Trimer

Construction of the Expression Plasmid pHiTe351

The expression plasmid pHiTe351 comprising the nucleotide sequence encoding the CBM1-trimer in operation connection with an Aspergillus promoter, signal sequence and Kex cleavage site and terminator, and further comprising an amdS gene for amdS selection in Aspergillus. The 0.57 kb region of CBM1-trimer gene was amplified from the plasmid pAT2486 by PCR with primer pairs:

SEQ ID NO: 24:
AGGATTTAGTCTTGATCGGATCCACCATGATGAAGTTCTTCACAACGATC
SEQ ID NO: 25:
CTATGCGTTATCGTACGCACCACGTGTTAAGGCTGACACTGCGAATAGAA

The obtained 0.57 kb DNA fragment was ligated into pHiTe169 (a derivative of pJaL1470 described in US 2017/0114091) by NEBuilder® HiFi DNA Assembly Master Mix according to the manufacture's protocol, to create pHiTe351.

(CBM1-trimer coding sequence)
SEQ ID NO: 26
atgatgaagttcttcacaacgatcctctcgactgcatcgctcgtcgcagccctccctg-
cagccgtcgattcgaaccacacgcctgcggcaccggaactcgtcgccaggtcccctatccgacgccagcagtcgctctacggtcagtgtggcggt
aacggatggtcgggaccgaccgagtgtacagcaggcgcatgtt-
gtcaggtccagaacccctggtattcgcagtgtttgcctgagccgacaccggagcctactcagtcgcctatctggggacagtgtggaggcaacggtt
ggacgggtgcaaccacgtgtcagtcgggactcaagtgtgagaaggtgaacgattggtactac-
cagtgtgtccctggcgcaacttcgcctggtggctcctccggatcccagtcctgttcgaacggcgtctgggcacagtgtggcggtcagaactggtcc
ggcaccccttgttgtacttcgggcaacacatgtgtcaagatcaacgatttctattcgcagtgtcagccttaa
(CBM1-trimer amino acid sequence)
SEQ ID NO: 27
MMKFFTTILSTASLVAALPAAVDSNHTPAAPELVARSPIRRQQSLYGQCGGNGWSGPTECTA-
GACCQVQNPWYSQCLPEPTPEPTQSPIWGQCGGNGVVTGATTCQSGLKCEKVNDWYYQCVP
GATSPGGSSGSQSCSNGVWAQCGGQNWSGTPCCTSGNTCVKINDFYSQCQP

CBM1-trimer in A. niger Strain

Chromosomal insertion into A. niger C4922 (a derivative of NN059461 which is described in US 2017/0114091) of the CBM1-trimer gene with amdS selective marker (pHiTe351) was performed as described in WO 2012/160093. Strains which grew well were purified and subjected to southern blotting analysis to confirm whether the CBM1-trimer gene was introduced at NA1, NA2, SP288 or PAY loci correctly or not. The following set of primers to make non-radioactive probe was used to analyze the selected transformants.

For the promoter region:

SEQ ID NO 28:
AAGGGATGCAAGACCAAACC
SEQ ID NO 29:
TGAAGAATTTGTGTTGTCTGAG

Genomic DNA extracted from the selected transformants was digested by SpeI and MluI, then probed with the promoter region. By the right gene introduction event, hybridized signals at the size of 3.6 kb (NA1), 4.4 kb (NA2), 2.4 kb (SP288) and 3.1 kb (PAY) by SpeI and MluI digestion was observed probed described above.

Among the strains given the right integration events of 4-copies of the genes at NA1, NA2, SP288 and PAY loci, one strain with CBM1-trimer was selected.

### CBM1-Trimer Expression in Shake Flask Fermentation.

Shake flasks containing 100 ml of the seed medium MSS (70 g Sucrose, 100 g Soybean powder (pH 6.0), water to 1 litre) were inoculated with spores from the A. niger strains and incubated at 30° C., with shaking (220 rpm) for 3 days. Ten ml of the seed culture was transferred to shake flasks containing 100 ml of the main medium MU-1 glu (260 g of glucose, 3 g of MgSO₄-7H2O, 5 g of KH₂PO₄, 6 g of K₂SO₄, amyloglycosidase trace metal solution 0.5 ml and urea 2 g (pH 4.5), water to 1 litre) and incubated at 30° C., with shaking (220 rpm) for 6 days. The culture supernatants were collected by centrifugation and used for sub-sequent purification. The expression of the intact CBM1-trimer was confirmed by SDS-PAGE analysis.

### Recovery of CBM1-Trimer.

After growing the transformed Aspergillus in shake flasks, the CBM1-trimer was purified from the supernatant using affinity chromatography as follows. The 25 ml culture supernatant was mixed with same volume of 0.1M sodium phosphate, 0.5M sodium chloride, pH 7.5 and then loaded onto Avicel (Fluka) packed in a chromatography column (column volume=8 ml). After washing with 3× column volumes of the same buffer, the target protein was eluted with 10× column volumes of milliQ water. The eluted fractions (judged by absorbance at 280 nm) were concentrated by ultrafiltration, and then the concentration was measured by its absorbance at 280 nm and SDS-PAGE.

The N-terminal sequence of the CBM1-trimer was further determined by MS spectrometry analysis.

(CBM1-trimer mature protein)
SEQ ID NO: 30
SPIRRQQSLYGQCGGNGWSGPTECTAGACCQVQNPWYSQCLPEPTPEPTQSPIWGQCGG-
NGVVTGATTCQSGLKCEKVNDWYYQCVPGATSPGGSSGSQSCSNGVWAQCGGQNWSGTPC
CTSGNTCVKINDFYSQCQP

The mature protein without the SPIRR (SEQ ID NO: 31)-terminus is

(SEQ ID NO: 32)
QQSLYGQCGGNGWSGPTECTAGACCQVQNPWYSQCLPEPTPEPTQSPIWGQCGG-
NGVVTGAT-
TCQSGLKCEKVNDWYYQCVPGATSPGGSSGSQSCSNGVWAQCGGQNWSGTPCCTSGNTC
VKINDFYSQCQP

### Example 3. Preparation of CBM1-Tetramer

CBM1-tetramer was derived by joining 4 polypeptides with linking amino acid sequences to form a composite polypeptide with 4 CBM1 units. The CBM1-tetramer comprised the same CBM1 components as for the CBM1-trimer of Example 2 in the same order, and using linkers derived from the linker to the starch binding domain from amyloglucosidase of Athelia rolfsii.
Between unit 1 and unit 2 the linker was encoded by the nucleotide sequence:

(SEQ ID NO: 33)
GGCGCCACCTCCCCCGGTGGTAGCTCCGGTTCT

Between unit 2 and unit 3 the linker was encoded by the nucleotide sequence:

(SEQ ID NO: 34)
GGCGCAACTTCGCCTGGTGGCTCCTCCGGATCC

Between unit 3 and unit 4 the linker was encoded by the nucleotide sequence:

(SEQ ID NO: 35)
GGTGCAACCTCACCTGGAGGTTCAAGCGGCTCA

All linkers had the amino acid sequence:

(SEQ ID NO: 36)
GATSPGGSSGS

The fourth unit of the tetramer was derived from the Endo-1,4-beta-glucanase GH45A from Neurospora tetrasperma and was encoded by the nucleotide sequence:
TGTACAGCGGATAAGTACGCGCAGTGTGGTGGCTCCgtacgtgtcttctttttttttgcttgttctacctcgcgcctcagtacaagagatactaattgatttagGGATGGTCCGGCTGTACGAACTGTCCTTCGGG ATCGACTTGTAAGACCATCAACGACTACTATCATCAGTGTGCATAA (the sequence contains an intron marked by lower-case letters) (SEQ ID NO: 22)
and had the amino acid sequence:

(SEQ ID NO: 23)
CTADKYAQCGGSGWSGCTNCPSGSTCKTINDYYHQCA

This provides the CBM1-tetramer molecule having nucleotide sequence:
CAGCAGTCGCTCTACGGTCAGTGTGGCGGTAACGGATGGTCGGGACCGACCGAGTGTACAG-CAGGCGCATGTTGTCAGGTCCAGAACCCCTGGTATTCGCAGTGTTTGGGCGCCACCTCCC CCGGTGGTAGCTCCGGTTCTCAGTCGCCTATCTGGGGACAGTGTGGAGGCAACGGTTGGAC-GGGTGCAACCACGTGTCAGTCGGGACTCAAGTGTGAGAAGGTGAACGATTGGTACTACCA GTGTGTCCCTGGCGCAACTTCGCCTGGTGGCTCCTCCGGATCCCAGTCCTGTTCGAACGGCGTCTGGGCACAGTGTGGCGGTCAGAACTGGTCCGGCACCCCTTGTTGTACTTCGGG CAACACATGTGTCAAGATCAACGATTTCTATTCGCAGTGTCAGCCTGGTGCAACCTCACCTG-GAGGTTCAAGCGGCTCATGTACAGCGGATAAGTACGCGCAGTGTGGTGGCTCCgtacgtgtctt ctttttttttgcttgttctacctcgcgcctcagtacaagagatactaattgatttagGGATGGTCCGGCTGTACGAACTGTCCTTCGGGATCGACTTGTAAGACCATCAACGACT ACTATCATCAGTGTGCA (the sequence contains an intron marked by lower-case letters) (SEQ ID NO: 37)
and the amino acid sequence:

(SEQ ID NO: 38)
QQSLYGQCGGNGWSGPTECTAGACCQVQNPWYSQCLGATSPGGSSGSQSPIWGQCGG-
NGVVTGATTCQSGLKCEKVNDWYYQCVPGATSPGGSSGSQSCSNGVWAQCGGQNWSGTPC
CTSGNTCVKINDFYSQCQPGATSPGGSSGSCTADKYAQCGGSGWSGCTNCPSGSTCK-
TINDYYHQCA

### Example 4

CBM-Multimer Anti-Crease Properties with Mixed Soil from Soil Ballast Evaluated on Cotton T-Shirts

White T-shirts for children produced in India were purchased from Decathlon, France. T-shirts were used as tracers for wrinkle count. 4 pieces of soil-ballast (SBL-CFT) in size 40×20 cm² equalizing 8g soil were added to each European front loader Full Scale Wash (FSW) machine. For FSW was employed Miele Softtronic W5841 washing machine (Program: Cottons; Additional program: Short; Temperature: 30° C.; Centrifuge: 1600 rpm; Ballast: 600-700 g 100% cotton textile). A model detergent composition, Model B, was dosed 3.3 g/L. CBM1-trimer of SEQ ID NO: 30, produced as in Example 2, dosed 0.25 ppm was added to individual washing machines and laundered as described. Two independent replica of each FSW were conducted. From each machine T-shirts were line-dried for 24 h at room temperature. Fabric pieces were evaluated by scoring according to the Standard AATCC Three-Dimensional Smoothness Appearance Replicas by a panel consisting of 4 panelists (the panel set-up was as close to AATCC method 124 as possible). Panelists were asked to compare each swatch with the AATCC smoothness standards ranking from SA value=1 (very wrinkled standard) to SA value 5=(totally smooth standard). After evaluation, average and standard error across the panel scores was calculated for each condition.

| | Textile evaluated by AATCC Smoothness standards | |
|---|---|---|
| | Average SA-value according to AATCC +/− | |
| | stE on average | |
| Protein | −CBM-trimer | +CBM-trimer |
| CBM1-trimer | 1.6 +/− 0.1 | 2.6 +/− 0.1 |

Values specify the average SA value rank given by the panel according to the AATCC smoothness standards+/− StE.

1. A fusion polypeptide comprising at least two carbohydrate binding modules (CBMs) or fragments thereof, wherein the polypeptide has carbohydrate binding activity.

2. The polypeptide of claim 1, which is a non-naturally occurring multimer comprising at least two carbohydrate binding modules or fragments thereof.

3. The polypeptide of claim 1, comprising three or more CBMs, such as four or more CBMs, five or more CBMs, six or more CBMs, seven or more CBMs, eight of more CBMs, nine or more CBMs, ten or more CBMs, 11 or more CBMs, 12 or more CBMs, 13 or more CBMs, 14 or more CBMs, 15 or more CBMs, 16 or more CBMs, 17 or more CBMs, 18 or more CBMs, 19 or more CBMs, or even 20 CBMs.

4. The polypeptide of claim 1, wherein the at least two CBMs are the same or different and are each independently selected.

5. The polypeptide of claim 1, which is a heteromultimer.

6. The polypeptide of claim 1, wherein each CBM is independently selected among CBM family 1, 4, 17, 28, 30, 44, 72 and 79, and mixtures thereof; preferably wherein each CBM is a CBM family 1 CBM.

7. The polypeptide of claim 1, which is a polypeptide comprising three, four, or five CBMs, each from CBM Family 1; preferably comprising three different CBMs, each from CBM Family 1.

8. The polypeptide of claim 1, wherein the CBMs are joined by a linker region.

9. The polypeptide of claim 8, wherein the linker region is heterologous to each of the CBMs.

10. The polypeptide of claim 1, wherein each CBM is independently selected among polypeptides having at least 60% sequence identity to one of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 23 e.g. at least 70%, sequence identity, e.g. at least 80% sequence identity, e.g. at least 90% sequence identity; e.g. at least 95%, sequence identity, e.g. at least 96% sequence identity, e.g. at least 97% sequence identity; e.g. at least 98% sequence identity or at least 99% sequence identity, or even 100% sequence identity.

11. The polypeptide of claim 1, wherein each CBM is independently selected from a CBM having the amino acid sequence of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22 or having an amino acid sequence that deviates from one of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 23 by 1, 2, 3, 4, 5, 6, 7, 8 or 9 substitutions, insertions or deletions.

12. The polypeptide of claim 1, comprising four CBMs having the amino acid sequence of SEQ ID NO: 2, SEQ ID NO: 4, SEQ ID NO: 6 and SEQ ID NO: 23, or having an amino acid sequence that deviates from one of SEQ ID NO: 2, SEQ ID NO: 4, SEQ ID NO: 6 and SEQ ID NO: 23 by 1, 2, 3, 4, 5, 6, 7, 8 or 9 substitutions, insertions or deletions.

13. The polypeptide of claim 1, having at least 60% sequence identity, e.g., 70% sequence identity, e.g. at least 80% sequence identity, e.g. at least 90% sequence identity; e.g. at least 95%, sequence identity, e.g. at least 96% sequence identity, e.g. at least 97% sequence identity; e.g. at least 98% sequence identity or at least 99% sequence identity, or even 100% sequence identity to the polypeptide of SEQ ID NO: 30, SEQ ID NO: 32, or SEQ ID NO: 38.

14. A method for reducing wrinkles and/or providing increased anti-crease properties and/or providing improved ease of ironing and/or providing improved shape retention in a cleaning process of a fabric or textile, the method comprising administering a fusion polypeptide of claim 1 to the fabric or textile.

15. The method of claim 14 wherein the fusion polypeptide is administered to the fabric or textile by incorporation of the fusion polypeptide into a washing process.


SEQUENCE LISTING (38 sequences)

SEQ ID NO: 1 - DNA, 114 bp, Fusarium longipes
  CDS (1)..(111)
cagtcccccatctggggacagtgtggtggaaacggatggactggtgcaacaacatgtcagtccggactcaagtgtgagaaagtgaacgattggtactaccagtgtgtcccctaa

SEQ ID NO: 2 - protein, 37 aa, Fusarium longipes
QSPIWGQCGGNGWTGATTCQSGLKCEKVNDWYYQCVP

SEQ ID NO: 3 - DNA, 144 bp, Fusarium longipes
  CDS (1)..(141)
gcaccggtcgaagaacgacagtcgtgttcgaacggagtctgggcacagtgtggtggtcagaactggtcgggtacaccctgttgtacatccggcaacacatgtgtcaaaatcaacgacttctactcgcagtgtcagcctggctaa

SEQ ID NO: 4 - protein, 47 aa, Fusarium longipes
APVEERQSCSNGVWAQCGGQNWSGTPCCTSGNTCVKINDFYSQCQPG

SEQ ID NO: 5 - DNA, 123 bp, Aspergillus clavatus
  CDS (1)..(120)
cagcagtccctctatggccagtgtggaggtaacggctggtccggacccacagagtgtacagcaggagcatgttgtcaggtccagaacccgtggtattcccagtgtctccctggcgattgttaa

SEQ ID NO: 6 - protein, 40 aa, Aspergillus clavatus
QQSLYGQCGGNGWSGPTECTAGACCQVQNPWYSQCLPGDC

SEQ ID NO: 7 - DNA, 381 bp, Ruminococcus flavefaciens
  CDS (1)..(381)
gatggttacaccattaagcccaacaagaaagtcacttactcggcactcggcgaagatgaacggatgattggcttctcgtacaaggacttcggcatctcctcgtcggaaaagatcacagaggtccaggtcaacatttcggccaacaagaacattggtaagtacgtcggccagttcggcacgtccacaaccgactcggcaaacggatactgggccatgggcgacgagatcactcagtccatctcgggtaactccggcacgatcacatggaaggtcccctcggatatctcgtcgatcatccagacgcagtatggcggagaaatcaaattcggagtgtggtggatcgattgtgatgagttcacaatcgattcggtggtcctcaaa

SEQ ID NO: 8 - protein, 127 aa, Ruminococcus flavefaciens
DGYTIKPNKKVTYSALGEDERMIGFSYKDFGISSSEKITEVQVNISANKNIGKYVGQFGTSTTDSANGYWAMGDEITQSISGNSGTITWKVPSDISSIIQTQYGGEIKFGVWWIDCDEFTIDSVVLK

SEQ ID NO: 9 - DNA, 567 bp, Artificial Sequence
  Unidentified microorganism
  CDS (1)..(567)
ggctacaagtacccgacagccgacgatttcgaaatcgtgtatgacatctcgtacaacgacgagtggtccgaattgttcttgttcggctcgtgggacaggactgccgtcaacttgtcgggatacaagggcatccgcgtggagatggacaaggcctatggcaacaaactccagatcaaggtgtacggcgacaagaagtccggtaccgatttcaacgaacagtatgcccctctctccgatacatcggcctccacgacggtcgatttcgacacctcgattttgggctcgacgttctggggtgtcacgttgcagacgaactccggtgcattgaccgcgacactcaaagaggccaagttgatcaaggccgacggaaccgaggaacctgcctcggtgaccgcagcatggggatgtacagtgactgccaagtcgaccccgaaacctaccggcatccacgccatccagttgatcaaaaccgaagcagatggtgccatctataacctccagggccagagggtgcagaacccccagaagggtatctacattcagaacggcaagaaatacgtgatgaaa

SEQ ID NO: 10 - protein, 189 aa, Artificial Sequence
  Synthetic Construct
GYKYPTADDFEIVYDISYNDEWSELFLFGSWDRTAVNLSGYKGIRVEMDKAYGNKLQIKVYGDKKSGTDFNEQYAPLSDTSASTTVDFDTSILGSTFWGVTLQTNSGALTATLKEAKLIKADGTEEPASVTAAWGCTVTAKSTPKPTGIHAIQLIKTEADGAIYNLQGQRVQNPQKGIYIQNGKKYVMK

SEQ ID NO: 11 - DNA, 435 bp, Hungateiclostridium thermocellum
  CDS (1)..(435)
ggcactctcggaggattcactacctccggcaccaacgccacaggagtggtggtgaacacgaccgagaaggcattcaagggagagaggggactcaagtggacagtcacatcggagggtgagggtactgccgagttgaagttggatggaggcacaatcgtcgtccctggcacgactatgactttccggatctggattccctcgggtgcacctattgcagccatccagccttacatcatgcctcatacaccggattggtcggaggtcctctggaactcgacctggaagggatacacgatggtcaagacggatgattggaacgagattaccctcactctcccggaagacgtggaccccacttggcctcagcagatgggaattcaggtccagaccatcgacgaaggcgaattcacaatctacgtggatgcgatcgattgg

SEQ ID NO: 12 - protein, 145 aa, Hungateiclostridium thermocellum
GTLGGFTTSGTNATGVVVNTTEKAFKGERGLKWTVTSEGEGTAELKLDGGTIVVPGTTMTFRIWIPSGAPIAAIQPYIMPHTPDWSEVLWNSTWKGYTMVKTDDWNEITLTLPEDVDPTWPQQMGIQVQTIDEGEFTIYVDAIDW

SEQ ID NO: 13 - DNA, 552 bp, Clostridium cellulovorans
  CDS (1)..(552)
gataccacagtgtccaggaagctcatggatctcgaggtgttcaagtccgcatccattaccggctggtccggatcggcaggaggcgaactcgaggtggcatccgattcgaacttgcccatcgacacatccgcaacctacaacggcttgccttccttgaggttgaacgtcacaaaggcctccgcacagtggtggtcctcgctcctcacactcaggggatggtgtactcaggacttgacacagtacctcgccaacggctatttggagttcaacgtgaaaggtaaggtcggaggcgaggacttccagattggactccaggaccagactcatgaacgagcagccggagactcggtcacctccgtgaagtcgatcaagaactatgtcaacatctcgaccaactggcagcacgtgaagatccccttgaaggacattatgggaccctccactggattcgacccgactacagcccgatgtatcaacatcgtgaagggctcctcggagatcttcacggcatggatcaacgacctcaagatcacgtcgacggacaacgagaag

SEQ ID NO: 14 - protein, 184 aa, Clostridium cellulovorans
DTTVSRKLMDLEVFKSASITGWSGSAGGELEVASDSNLPIDTSATYNGLPSLRLNVTKASAQWWSSLLTLRGWCTQDLTQYLANGYLEFNVKGKVGGEDFQIGLQDQTHERAAGDSVTSVKSIKNYVNISTNWQHVKIPLKDIMGPSTGFDPTTARCINIVKGSSEIFTAWINDLKITSTDNEK

SEQ ID NO: 15 - DNA, 1083 bp, Clostridium cellulovorans
  CDS (1)..(1083)
ttgtgggatttcaacgacggcaccaagcagggcttcggcgtcaacggcgattcccctgtcgaagatgtggtcatcgagaacgaggcaggtgccttgaagctctccggcttggatgcctccaacgacgtctcggagggaaactactgggcaaacgcgaggctctcggcagatggatggggcaaatcggtggacattttgggtgcagagaaactcaccatggatgtcatcgtggacgaaccgacgaccgtctccattgcagccatccctcagggtccttccgcgaactgggtgaaccccaaccgtgccattaaggtcgagcccaccaacttcgtgcccttgggcgataagttcaaggcggaactcacaatcacgtccgcagactcgccttccctcgaagcaattgccatgcacgcagagaacaacaacatcaacaacatcatcctcttcgtgggaacggaaggtgccgacgtcatttacctcgataacatcaaagtgatcggtacagaagtggaaattcccgtggtccacgatcccaagggcgaggccgtgctcccctcggtcttcgaagatggtaccaggcagggatgggattgggcaggtgagtcgggtgtgaagactgccctcacaatcgaggaagccaacggatcgaacgcgctctcctgggaattcggctaccccgaagtcaaaccgtcggacaactgggcaacagcaccgaggctcgacttctggaagtcggacttggtgaggggagagaacgactacgtgactttcgacttctatctcgatcctgtgagggcaaccgaaggcgcaatgaacattaacctcgtcttccagcctcccacgaacggatactgggtgcaggcacctaaaacttacaccatcaacttcgatgagctcgaagaggccaaccaggtgaacggcttgtatcactacgaggtcaagattaacgtccgcgatatcacaaacatccaggacgacacactcttgaggaacatgatgatcatcttcgccgacgtcgaatcggatttcgcaggacgcgtcttcgtggacaacgtccggttcgagggagcagcgaccactgag

SEQ ID NO: 16 - protein, 361 aa, Clostridium cellulovorans
LWDFNDGTKQGFGVNGDSPVEDVVIENEAGALKLSGLDASNDVSEGNYWANARLSADGWGKSVDILGAEKLTMDVIVDEPTTVSIAAIPQGPSANWVNPNRAIKVEPTNFVPLGDKFKAELTITSADSPSLEAIAMHAENNNINNIILFVGTEGADVIYLDNIKVIGTEVEIPVVHDPKGEAVLPSVFEDGTRQGWDWAGESGVKTALTIEEANGSNALSWEFGYPEVKPSDNWATAPRLDFWKSDLVRGENDYVTFDFYLDPVRATEGAMNINLVFQPPTNGYWVQAPKTYTINFDELEEANQVNGLYHYEVKINVRDITNIQDDTLLRNMMIIFADVESDFAGRVFVDNVRFEGAATTE

SEQ ID NO: 17 - protein, 166 aa, Clostridium cellulovorans
LWDFNDGTKQGFGVNGDSPVEDVVIENEAGALKLSGLDASNDVSEGNYWANARLSADGWGKSVDILGAEKLTMDVIVDEPTTVSIAAIPQGPSANWVNPNRAIKVEPTNFVPLGDKFKAELTITSADSPSLEAIAMHAENNNINNIILFVGTEGADVIYLDNIKVI

SEQ ID NO: 18 - protein, 195 aa, Clostridum cellulovorans
GTEVEIPVVHDPKGEAVLPSVFEDGTRQGWDWAGESGVKTALTIEEANGSNALSWEFGYPEVKPSDNWATAPRLDFWKSDLVRGENDYVTFDFYLDPVRATEGAMNINLVFQPPTNGYWVQAPKTYTINFDELEEANQVNGLYHYEVKINVRDITNIQDDTLLRNMMIIFADVESDFAGRVFVDNVRFEGAATTE

SEQ ID NO: 19 - DNA, 456 bp, Cellulomonas fimi
  CDS (1)..(456)
gcatcgccgattggtgagggtaccttcgatgatggtcccgagggctgggtcgcatatggtaccgatggtcccttggatacgtcgacaggagcactctgtgtcgcagtgcctgcaggttccgcacagtacggtgtcggagtggtcttgaacggagtcgcaatcgaggagggtaccacgtatacgctccgttatactgcaaccgcatccacggatgtcacagtccgagcactcgtgggacagaacggagcaccctatggaactgtcctcgatacatcgcctgcactcacatccgaacctcgacaggtcaccgaaaccttcacagcatcggcaacttatcctgcaacgcctgcagcagatgatcctgagggtcagatcgcattccagttgggaggcttctcggcagatgcatggaccttctgtttggatgatgtcgcattggattcggaggtcgagttg

SEQ ID NO: 20 - protein, 152 aa, Cellulomonas fimi
ASPIGEGTFDDGPEGWVAYGTDGPLDTSTGALCVAVPAGSAQYGVGVVLNGVAIEEGTTYTLRYTATASTDVTVRALVGQNGAPYGTVLDTSPALTSEPRQVTETFTASATYPATPAADDPEGQIAFQLGGFSADAWTFCLDDVALDSEVEL

SEQ ID NO: 21 - protein, 19 aa, Artificial Sequence
  Signal peptide sequence
MKLSWLVAAALTAASVVSA

SEQ ID NO: 22 - DNA, 182 bp, Neurospora tetrasperma
  CDS (1)..(36)
  Intron (37)..(104)
  CDS (105)..(179)
tgtacagcggataagtacgcgcagtgtggtggctccgtacgtgtcttctttttttttgcttgttctacctcgcgcctcagtacaagagatactaattgatttagggatggtccggctgtacgaactgtccttcgggatcgacttgtaagaccatcaacgactactatcatcagtgtgcataa

SEQ ID NO: 23 - protein, 37 aa, Neurospora tetrasperma
CTADKYAQCGGSGWSGCTNCPSGSTCKTINDYYHQCA

SEQ ID NO: 24 - DNA, 50 bp, Artificial Sequence
  Synthetic Construct
aggatttagtcttgatcggatccaccatgatgaagttcttcacaacgatc

SEQ ID NO: 25 - DNA, 50 bp, Artificial Sequence
  Synthetic Construct
ctatgcgttatcgtacgcaccacgtgttaaggctgacactgcgaatagaa

SEQ ID NO: 26 - DNA, 522 bp, Artificial Sequence
  Synthetic Construct
atgatgaagttcttcacaacgatcctctcgactgcatcgctcgtcgcagccctccctgcagccgtcgattcgaaccacacgcctgcggcaccggaactcgtcgccaggtcccctatccgacgccagcagtcgctctacggtcagtgtggcggtaacggatggtcgggaccgaccgagtgtacagcaggcgcatgttgtcaggtccagaacccctggtattcgcagtgtttgcctgagccgacaccggagcctactcagtcgcctatctggggacagtgtggaggcaacggttggacgggtgcaaccacgtgtcagtcgggactcaagtgtgagaaggtgaacgattggtactaccagtgtgtccctggcgcaacttcgcctggtggctcctccggatcccagtcctgttcgaacggcgtctgggcacagtgtggcggtcagaactggtccggcaccccttgttgtacttcgggcaacacatgtgtcaagatcaacgatttctattcgcagtgtcagccttaa

SEQ ID NO: 27 - protein, 173 aa, Artificial Sequence
  Synthetic Construct
  SIGNAL (1)..(36)
  mat_peptide (37)..(173)
MMKFFTTILSTASLVAALPAAVDSNHTPAAPELVARSPIRRQQSLYGQCGGNGWSGPTECTAGACCQVQNPWYSQCLPEPTPEPTQSPIWGQCGGNGWTGATTCQSGLKCEKVNDWYYQCVPGATSPGGSSGSQSCSNGVWAQCGGQNWSGTPCCTSGNTCVKINDFYSQCQP

SEQ ID NO: 28 - DNA, 20 bp, Artificial Sequence
  Synthetic Construct
aagggatgcaagaccaaacc

SEQ ID NO: 29 - DNA, 22 bp, Artificial Sequence
  Synthetic Construct
tgaagaatttgtgttgtctgag

SEQ ID NO: 30 - protein, 137 aa, Artificial Sequence
  Synthetic Construct
SPIRRQQSLYGQCGGNGWSGPTECTAGACCQVQNPWYSQCLPEPTPEPTQSPIWGQCGGNGWTGATTCQSGLKCEKVNDWYYQCVPGATSPGGSSGSQSCSNGVWAQCGGQNWSGTPCCTSGNTCVKINDFYSQCQP

SEQ ID NO: 31 - protein, 5 aa, Artificial Sequence
  Synthetic Construct
SPIRR

SEQ ID NO: 32 - protein, 132 aa, Artificial Sequence
  Synthetic Construct
QQSLYGQCGGNGWSGPTECTAGACCQVQNPWYSQCLPEPTPEPTQSPIWGQCGGNGWTGATTCQSGLKCEKVNDWYYQCVPGATSPGGSSGSQSCSNGVWAQCGGQNWSGTPCCTSGNTCVKINDFYSQCQP

SEQ ID NO: 33 - DNA, 33 bp, Artificial Sequence
  Synthetic Construct
ggcgccacctcccccggtggtagctccggttct

SEQ ID NO: 34 - DNA, 33 bp, Artificial Sequence
  Synthetic Construct
ggcgcaacttcgcctggtggctcctccggatcc

SEQ ID NO: 35 - DNA, 33 bp, Artificial Sequence
  Synthetic Construct
ggtgcaacctcacctggaggttcaagcggctca

SEQ ID NO: 36 - protein, 33 aa, Artificial Sequence
  Synthetic Construct
GGTGCAACCTCACCTGGAGGTTCAAGCGGCTCA

SEQ ID NO: 37 - DNA, 617 bp, Artificial Sequence
  Synthetic Construct
  exon (1)..(474)
  Intron (475)..(542)
  exon (543)..(617)
cagcagtcgctctacggtcagtgtggcggtaacggatggtcgggaccgaccgagtgtacagcaggcgcatgttgtcaggtccagaacccctggtattcgcagtgtttgggcgccacctcccccggtggtagctccggttctcagtcgcctatctggggacagtgtggaggcaacggttggacgggtgcaaccacgtgtcagtcgggactcaagtgtgagaaggtgaacgattggtactaccagtgtgtccctggcgcaacttcgcctggtggctcctccggatcccagtcctgttcgaacggcgtctgggcacagtgtggcggtcagaactggtccggcaccccttgttgtacttcgggcaacacatgtgtcaagatcaacgatttctattcgcagtgtcagcctggtgcaacctcacctggaggttcaagcggctcatgtacagcggataagtacgcgcagtgtggtggctccgtacgtgtcttctttttttttgcttgttctacctcgcgcctcagtacaagagatactaattgatttagggatggtccggctgtacgaactgtccttcgggatcgacttgtaagaccatcaacgactactatcatcagtgtgca

SEQ ID NO: 38 - protein, 183 aa, Artificial Sequence
  Synthetic Construct
QQSLYGQCGGNGWSGPTECTAGACCQVQNPWYSQCLGATSPGGSSGSQSPIWGQCGGNGWTGATTCQSGLKCEKVNDWYYQCVPGATSPGGSSGSQSCSNGVWAQCGGQNWSGTPCCTSGNTCVKINDFYSQCQPGATSPGGSSGSCTADKYAQCGGSGWSGCTNCPSGSTCKTINDYYHQCA